(19) United States
(12) Patent Application Publication
(10) Pub. No.: US 2026/0248775 A1
(43) Pub. Date: Aug. 27, 2026

(54) NEW INHIBITORS OF PHOSPHATIDYLINOSITOL 3-KINASE

(71) Applicants: INSTITUT NATIONAL DE LA SANTÉ ET DE LA RECHERCHE MÉDICALE, PARIS (FR); CENTRE NATIONAL DE LA RECHERCHE SCIENTIFIQUE, PARIS CEDEX 16 (FR); ASSISTANCE PUBLIQUE — HOPITAUX DE PARIS, PARIS (FR); UNIVERSITE PARIS CITE, PARIS (FR)

(72) Inventors: Laurent MICOUIN, PARIS (FR); Serge TURCAUD, PARIS (FR); Guillaume CANAUD, PARIS (FR); Quitterie VENOT, PARIS (FR)

(73) Assignees: INSTITUT NATIONAL DE LA SANTÉ ET DE LA RECHERCHE MÉDICALE, PARIS (FR); CENTRE NATIONAL DE LA RECHERCHE SCIENTIFIQUE, PARIS CEDEX 16 (FR); ASSISTANCE PUBLIQUE — HOPITAUX DE PARIS, PARIS (FR); UNIVERSITE PARIS CITE, PARIS (FR)

(21) Appl. No.: 19/125,101

(22) PCT Filed: Oct. 27, 2023
(86) PCT No.: PCT/EP2023/080137

§ 371 Date: Apr. 28, 2025

(30) Foreign Application Priority Data
Oct. 28, 2022 (EP) 22306632.5

(51) Int. Cl. A61K 31/4439; A61P 13/12; A61P 37/06; C07D 417/14

(52) U.S. Cl. CPC A61K 31/4439; A61P 13/12; A61P 37/06; C07D 417/14

(57) ABSTRACT

The prevention and/or treatment of protein tyrosine kinase mediated diseases, in particular phosphatidylinositol 3-kinases (PI3Ks) mediated diseases. PI3Ks are well-known as oncology targets and several inhibitors of PI3Ks have been developed that inhibit multiple class 1A PI3K isoforms. The development of selective inhibitors of PI3K-α may enable sufficient target inhibition while avoiding some toxicity drawbacks known for pan PI3K inhibitors. New specific carbamothioyl-pyrrolidine-carboxamide compounds of formula (I) exhibit an advantageous PI3K inhibitory activity, especially with a high selectivity for the isoform PI3K alpha. In particular, compounds of formula (I), or a deuterated or tritiated form of the compound of formula (I), and pharmaceutically acceptable salts thereof:

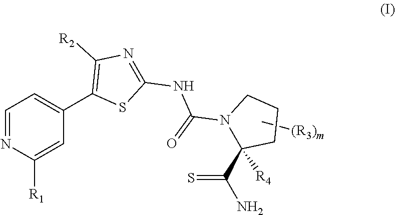

Also, a pharmaceutical composition containing the compound of formula (I) and the compounds of formula (I) for use in the treatment and/or prevention of Protein tyrosine kinase mediated diseases.

### FIELD OF THE INVENTION

The present invention relates to new specific carbamothioyl-pyrrolidine-carboxamide compounds, to their synthesis process and to their therapeutic use.

The compounds described herein are new phosphatidylinositol 3-kinase (PI3K) inhibitors, especially alpha-selective phosphatidylinositol 3-kinase (PI3K alpha) inhibitors. The compounds described herein are thus useful in the prevention and/or treatment of protein tyrosine kinase mediated diseases, in particular PI3K mediated diseases.

### BACKGROUND OF THE INVENTION

Phosphatidylinositol 3-kinases (PI3Ks) comprise a family of lipid kinases that phosphorylate phosphatidylinositides at the 3′ position of the inositol ring. They have been divided into three classes based on their substrate specificity and sequence homology.

Class I PI3Ks phosphorylate phosphatidylinositol-4,5-diphosphate (PIP₂) downstream of either receptor tyrosine kinases or G-protein coupled receptors to form the second messenger phosphatidylinositol-3,4,5-trisphosphate (PIP₃), which signals for increased cell growth, metabolism, and cell-cycle progression.

Class I consists of four family members, each of which forms a heterodimer between a catalytic subunit, p110, and a regulatory subunit. The family is further subdivided into Class IA, where isoforms p110α, p110β and p110δ form heterodimers with the p85 family of regulatory subunits, and Class IB, where p110γ is the sole member and forms a heterodimer with either the p87 or p101 regulatory subunit.

Each isoform has been shown to have overlapping, but non-redundant physiological roles. PI3K-α and β are both ubiquitously expressed. PI3K-α plays a key role in glucose homeostasis and insulin signaling and is involved in driving myocardial growth via PIP₃ dependent pathways. PI3K-β, in contrast, has been shown to regulate the activity of the platelet integrin α_{IIb}β₃ in the context of platelet adhesion and aggregation. As such PI3K-β is under investigation as a novel treatment for thrombosis, and the first in-human trials have shown promising results. PI3K-γ and δ have more restricted expression, largely limited to the hematopoietic system. They both play important, non-redundant roles in the immune system, and so are both under consideration as immune modulatory targets. PI3K-γ inhibition is being pursued for rheumatoid arthritis and asthma, and PI3K-δ for activated PI3K-δ syndrome (APDS).

Their diverse functions notwithstanding, PI3Ks are perhaps most well-known as oncology targets. The PI3K pathway is one of the most frequently dysregulated in cancer.

Thus, several inhibitors of PI3Ks have been developed that inhibit multiple class 1A PI3K isoforms and commonly known as “pan-PI3K” inhibitors.

The development of isoform selective inhibitors is vital to uncovering the unique functions of each isoform and their corresponding therapeutic potential. Significant progress has been made, and isoform selective inhibitors are now available for each of the four Class I isoforms. They continue to be useful in uncovering important details of PI3K physiology and the understanding of cancer signaling.

In particular, oncogenic mutations in the gene encoding the p110α catalytic subunit, PIK3CA, are common in breast, colon, and endometrial cancers. Somatic, missense mutations have been identified throughout the sequence of p110α. Interestingly, about 80% of these mutations are concentrated at 3 ‘hotspots’, Glu542Lys, Glu545Lys and His1047Arg.

Furthermore, the development of selective inhibitors of PI3K-α may enable sufficient target inhibition while avoiding some toxicity drawbacks known for pan PI3K inhibitors.

Thus, some selective inhibitors of PI3K-α have been developed, such as the 2-carboxamide cycloamino urea derivatives disclosed in application WO 2010/029082.

Document WO 2017/001362 relates to the treatment of cancer with Taselisib® which displays greater selectivity for PI3K alpha isoform.

There is still a need to provide further compounds, suitable as PI3K inhibitors for treating and/or preventing proliferative diseases, such as cancers.

In particular, there is a need to provide further compounds able to selectively inhibit the isoform PI3K alpha, advantageously with higher selectivity and/or higher activity.

### SUMMARY OF THE INVENTION

The inventors have found that the compounds of formula (I) defined hereinafter exhibit an advantageous phosphatidylinositol 3-kinase (PI3K) inhibitory activity, in particular towards the isoform alpha.

Further, as demonstrated in the examples below, these compounds advantageously show an improved selectivity for PI3K alpha, with respect to beta and/or delta and/or gamma subtypes.

Hence, the compounds of formula (I) are suitable to be used in the treatment and/or the prevention of Protein tyrosine kinase mediated diseases, in particular PI3K mediated diseases, more particularly of diseases mediated by the isoform alpha of PI3K.

Advantageously, the compounds of formula (I) exhibit such activity and improved selectivity without inducing insulin resistance problems, that may be observed for some known selective PI3K-α inhibitors. In particular, the compounds of formula (I) cause few, or even do not cause, hyperglycemia.

Moreover, the compounds of formula (I) exhibit a prolonged inhibitory activity against tissue-based AKT.

The present invention therefore relates, according to a first of its aspects, to compounds of formula (I), as defined below.

The present invention further relates, according to another of its aspects, to a process for manufacturing these compounds and to specific intermediate compounds involved in such process, as detailed hereafter.

According to still another of its aspects, the present invention relates to a pharmaceutical composition comprising a compound of formula (I) as defined below, or a deuterated or tritiated form of the compound of formula (I), or a pharmaceutically acceptable salt thereof.

The present invention further relates to a compound of formula (I) as defined below, or a deuterated or tritiated form of the compound of formula (I), or a pharmaceutically acceptable salt thereof, for use as a medicament.

Another aspect of the disclosure relates to a compound of formula (I) as defined below for its use as PI3K inhibitor, especially as PI3K alpha inhibitor.

The present invention further relates to a compound of formula (I) as defined below, or a deuterated or tritiated form of the compound of formula (I), or a pharmaceutically acceptable salt thereof, for use in the treatment and/or prevention of Protein tyrosine kinase mediated diseases, in particular PI3K mediated diseases, more particularly of diseases mediated by PI3K alpha.

### BRIEF DESCRIPTION OF THE FIGURES

FIG. 1 (A to C) illustrates the ability of Compound 5 of the invention to improve kidney lesions in NZBWF1/J mouse model. A. Urinary albumin to creatinine ratio (Ualb/Cre) (Ordinate, mg/mmol). Abscissa (From left to right): Vehicle and Compound 5 (50 mg/kg). B. Blood urea nitrogen level (BUN) (Ordinate, mmol/L). Abscissa (From left to right): Vehicle and Compound 5 (50 mg/kg). C. Glomerular lesion score of kidneys 4 weeks after uninephrectomy followed by treatment with either the vehicle or Compound 5 (n=6 mice per group). AU: Arbitrary Unit (Ordinate). Abscissa (From left to right): Vehicle and Compound 5 (50 mg/kg).

FIG. 2 illustrates the variation of insulin over time for the groups of mice treated either with a vehicle or with Compound 5 of the invention. Ordinate: insulin (ng/ml). Abscissa: time (hours).

### DEFINITIONS

As used herein, the term “patient” refers to either an animal, such as a valuable animal for breeding, company or preservation purposes, or preferably a human or a human child, which is afflicted with, or has the potential to be afflicted with, one or more diseases and conditions described herein.

In particular, as used in the present application, the term “patient” refers to a mammal such as a rodent, cat, dog, primate or human, preferably said subject is a human and also extends to birds.

The identification of those patients who are in need of treatment of herein-described diseases and conditions is well within the ability and knowledge of one skilled in the art. A veterinarian or a physician skilled in the art can readily identify, by the use of clinical tests, physical examination, medical/family history or biological and diagnostic tests, those patients who are in need of such treatment.

In the context of the invention, the term “treating” or “treatment”, as used herein, means reversing, alleviating, inhibiting the progress of, or preventing medical conditions of patients suffering from the diseases as described herein.

As used herein, an “effective amount” refers to an amount of a compound of the present invention which is effective in preventing, reducing, eliminating, treating or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

The term “effective amount” includes “prophylaxis-effective amount” as well as “treatment-effective amount”.

The term “preventing”, as used herein, means reducing the risk of onset or slowing the occurrence of a given phenomenon, namely in the present invention, a disease as described herein.

As used herein, “preventing” also encompasses “reducing the likelihood of occurrence” or “reducing the likelihood of reoccurrence”.

The term “prophylaxis-effective amount” refers to a concentration of compound of this invention that is effective in inhibiting, preventing, decreasing the likelihood of the inflammatory disease, a disease caused by a virus, and more particularly a retrovirus or cancer.

Likewise, the term “treatment-effective amount” refers to a concentration of compound that is effective in treating a disease as described herein.

As used herein, the term “pharmaceutically acceptable” refers to those compounds, materials, excipients, compositions or dosage forms which are, within the scope of sound medical judgment, suitable for contact with the tissues of human beings and animals without excessive toxicity, irritation, allergic response or other problem complications commensurate with a reasonable benefit/risk ratio.

The term “prodrug” means a compound without biological activity which is convertible in vivo by metabolic means (e.g. by hydrolysis, reduction or oxidation) to a compound of the present invention. For example, an ester prodrug of a compound of the present invention may be convertible by hydrolysis in vivo to the parent molecule. Suitable esters of compounds of the present invention are for example acetates, citrates, lactates, tartrates, malonates, oxalates, salicylates, propionates, succinates, fumarates, maleates, methylene-bis-β-hydroxynaphthoates, gentisates, isethionates, di-p-toluoyltartrates, methanesulphonates, ethanesulphonates, benzenesulphonates, p-toluenesulphonates, cyclohexylsulfamates and quinates. Examples of ester prodrugs are those described by F. J. Leinweber, Drug Metab. Res., 1987, 18, 379. As used herein, references to the compounds of the present invention are meant to also include any prodrug or metabolite forms.

The term “inhibit” or “inhibition” in the context of enzymes, for example in the context of PI3K and more particularly PI3K-α, refers to a reduction in the activity of the enzyme.

As used herein, the term “PI3K” refers to phosphatidylinositol 3-kinases, sometimes also called PI3-kinases, PI(3) Ks, PI3Ks or PI3K(s). PI3K enzymes are a family of enzymes involved in cellular functions including, but not limited to cell growth, proliferation, differentiation, motility, survival and intracellular trafficking. PIK3CA is the gene that codes for the P110a protein which is also called PIK3CA or PIK3C-alpha protein.

As used herein, the expressions “Protein tyrosine kinase mediated diseases”, “PI3K mediated diseases” or “PI3K-α mediated diseases” more particularly refers to diseases associated with overexpression and/or aberrant activity of Protein tyrosine kinase, PI3K and/or PI3K-α respectively.

A “proliferative disease” refers to a disease that occurs due to abnormal growth or extensions by the multiplication of cells. Exemplary proliferative diseases include cancers, benign neoplasms, angiogenesis, inflammatory diseases including autoimmune diseases/disorders.

The term “cancer” refers to a class of diseases characterized by the development of abnormal cells that proliferate uncontrollably and have the ability to infiltrate and destroy normal body tissues. It includes malignant tumors and benign tumors, metastatic tumors and non-metastatic tumors, solid tumors and non-solid tumors, such as Blood-Related Cancers which may thus include Leukaemia, Lymphoma and Myeloma; it may also relate to Central Nervous System (CNS) cancers and non-CNS cancers. Unless stated otherwise, the term “cancer” also encompasses juvenile and non-juvenile cancers, Recurrent and Non-Recurrent cancers as well as cancer relapses.

In the context of the present invention, the term:
“halogen” is understood to mean chlorine, fluorine, bromine, or iodine, and in particular denotes chlorine, fluorine or bromine, preferably fluorine;
“(C₁-Cₓ)alkyl”, as used herein, respectively refers to a C₁-Cₓ normal, secondary or tertiary monovalent saturated, linear or branched, hydrocarbon radical, for example (C₁-C₆)alkyl. Examples are, but are not limited to, methyl, ethyl, propyl, n-propyl, isopropyl, butyl, isobutyl, sec-butyl, tert-butyl, pentyl, isopentyl, hexyl and isohexyl groups, and the like;
“(C₃-Cₓ)cycloalkyl”, as used herein, refers to a cyclic saturated hydrocarbon radical, comprising from 3 to x carbon atoms, saturated or partially unsaturated and unsubstituted or substituted. Examples are, but are not limited to, cyclopropyl, cyclobutyl, cyclopentyl and cyclohexyl;
“Pharmaceutically acceptable salt” refers to salts which are, within the scope of sound medical judgment, suitable for use in contact with the tissues of humans and lower animals without undue toxicity, irritation, allergic response, and the like. Pharmaceutically acceptable salts include those derived from suitable organic and inorganic acids or bases.

Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺[(C₁-C₄)alkyl]₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The compounds of formula (I) or any of their pharmaceutically acceptable salts may form solvates or hydrates and the invention includes all such solvates and hydrates.

The terms “hydrates” and “solvates” simply mean that the compounds (I) according to the invention can be in the form of a hydrate or solvate, i.e. combined or associated with one or more water or solvent molecules. This is only a chemical characteristic of such compounds, which can be applied for all organic compounds of this type.

In the context of the invention, compounds of formula (I) as defined herein may also include deuterium or tritium. Deuterated or tritiated forms of the compounds simply means that hydrogen atoms (H) in these compounds can be partially or totally replaced by deuterium (D) or tritium (T) atoms.

The compounds of formula (I) can comprise one or more asymmetric carbon atoms. They can thus exist in the form of enantiomers or of diastereoisomers. These enantiomers, diastereoisomers and their mixtures, are encompassed within the scope of the present invention.

The compounds of formula (I) may be under amorphous or crystalline forms, which are encompassed within the scope of the present invention as well.

### DETAILED DESCRIPTION OF THE INVENTION

The present invention relates to compounds of formula (I), or a deuterated or tritiated form of the compound of formula (I), or pharmaceutically acceptable salts thereof:

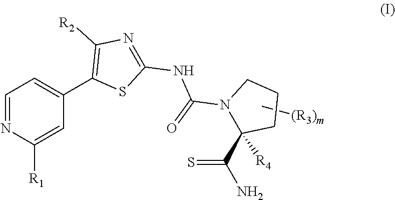

wherein:
R₁ is a (C₁-C₆)alkyl group or a (C₃-C₆) cycloalkyl group, unsubstituted or substituted with one or more fluorine atoms;
R₂ is chosen from:
   a hydrogen atom, and
   a (C₁-C₆)alkyl group,
m is 0, 1 or 2;
each R₃, when present, is independently chosen from:
   a fluorine atom,
   a (C₁-C₆)alkyl group, unsubstituted or substituted with one or more halogen atoms,
   a (C₃-C₆) cycloalkyl group, unsubstituted or substituted with one or more halogen atoms,
   a hydroxy group,
   a (C₁-C₆)alkoxy group, and
   a —NRR′ group, R and R′ being independently chosen from a hydrogen atom and a (C₁-C₆)alkyl group,
      or two R₃, that are borne by the same carbon atom, form with the carbon atom bearing them a (C₃-C₆) cycloalkyl ring, unsubstituted or substituted with one or more fluorine atoms; and
R₄ is chosen from:
   a fluorine atom,
   a hydrogen atom,
   a (C₁-C₆)alkyl group, unsubstituted or substituted with one or more halogen atoms, and
   a (C₃-C₆) cycloalkyl group, unsubstituted or substituted with one or more halogen atoms,
      or R₃ and R₄, when they are borne by two adjacent carbon atoms, form with the carbon atoms bearing them a (C₃-C₆) cycloalkyl ring, unsubstituted or substituted with one or more halogen atom.

Among the compounds of formula (I), mention may be made of the compounds for which R₁ is a (C₁-C₆)alkyl group, in particular a (C₁-C₄)alkyl group, and more particularly a tert-butyl group, unsubstituted or substituted with one or more fluorine atoms.

In an embodiment, in the compounds of formula (I), the R₁ group is a tert-butyl group substituted with one to three fluorine atoms.

In another embodiment, in the compounds of formula (I), the R₁ group is an unsubstituted tert-butyl group.

Among the compounds of formula (I), mention may be made of the compounds for which R₂ is a (C₁-C₆)alkyl group, in particular a (C₁-C₄)alkyl group and more particularly a methyl group.

In an embodiment, mention may be made of the compounds of formula (I) for which m is 0.

In another embodiment, mention may be made of the compounds of formula (I) for which m is 1 or 2, in particular for which m is 1.

In an embodiment, in the compounds of formula (I), m is 1 or 2, and each R₃ is independently chosen from:
a fluorine atom,
a (C₁-C₆)alkyl group, in particular a (C₁-C₄)alkyl group and more particularly a methyl group, unsubstituted or substituted with one or more halogen atoms,
a (C₃-C₆) cycloalkyl group unsubstituted or substituted with one or more halogen atoms, especially one or more fluorine atom;
a hydroxy group,
a (C₁-C₆)alkoxy group, in particular a (C₁-C₄)alkoxy group and more particularly a methoxy group, and
a —NH₂ group.

In an embodiment, in the compounds of formula (I), m is 1 or 2, and each R₃ is independently chosen from:
a fluorine atom,
a (C₁-C₆)alkyl group, in particular a (C₁-C₄)alkyl group and more particularly a methyl group, unsubstituted or substituted with one or more halogen atoms; for example a methyl group or a trifluoromethyl group;
a (C₃-C₆) cycloalkyl group unsubstituted or substituted with one or more halogen atoms, especially one or more fluorine atom;
a hydroxy group, and
a (C₁-C₆)alkoxy group, in particular a (C₁-C₄)alkoxy group and more particularly a methoxy group.

In a particular embodiment, m is 1 or 2, and each R₃ is independently chosen from:
a fluorine atom,
a (C₁-C₆)alkyl group, in particular a (C₁-C₄)alkyl group and more particularly a methyl group, unsubstituted or substituted with one or more halogen atoms; for example a methyl group or a trifluoromethyl group;
a hydroxy group, and
a (C₁-C₆)alkoxy group, in particular a (C₁-C₄)alkoxy group and more particularly a methoxy group.

In another embodiment, in the compounds of formula (I), m is 2 and two R₃, that are borne by the same carbon atom, form with the carbon atom bearing them a (C₃-C₆) cycloalkyl ring, in particular a cyclopropyl ring, unsubstituted or substituted with one or more fluorine atoms.

Among the compounds of formula (I), mention may be made of the compounds for which R₄ is chosen from:
a hydrogen atom, and
a (C₁-C₆)alkyl group, in particular a (C₁-C₄)alkyl group and more particularly a methyl group, unsubstituted or substituted with one or more halogen atoms, in particular with one or more fluorine atoms.

All these sub-groups and specific embodiments, taken alone or in combination, are part of the description.

In an embodiment, mention may be made of the compounds of formula (I) for which:
R₁ is a (C₁-C₆)alkyl group or a (C₃-C₆) cycloalkyl group, unsubstituted or substituted with one or more fluorine atoms;
R₂ is chosen from:
   a hydrogen atom, and
   a (C₁-C₆)alkyl group;
m is 0, 1 or 2;
each R₃, when present, is independently chosen from:
   a fluorine atom,
   a (C₁-C₆)alkyl group, unsubstituted or substituted with one or more halogen atoms,
   a (C₃-C₆) cycloalkyl group unsubstituted or substituted with one or more halogen atoms,
   a hydroxy group, and
   a (C₁-C₆) alkoxy group,
      or two R₃, that are borne by the same carbon atom, form with the carbon atom bearing them a (C₃-C₆) cycloalkyl ring, unsubstituted or substituted with one or more fluorine atoms; and
R₄ is chosen from:
   a fluorine atom,
   a hydrogen atom, and
   a (C₁-C₆)alkyl group, unsubstituted or substituted with one or more halogen atoms.

In another embodiment, mention may be made of the compounds of formula (I) for which:
R₁ is a (C₁-C₆)alkyl group, in particular a (C₁-C₄)alkyl group, unsubstituted or substituted with one to three fluorine atoms; and more particularly a tert-butyl group, unsubstituted or substituted with one to three fluorine atoms;
R₂ is a (C₁-C₆)alkyl group, in particular a (C₁-C₄)alkyl group and more particularly a methyl group,
m is 0, 1 or 2;
each R₃, when present, is independently chosen from:
   a fluorine atom,
   a (C₁-C₆)alkyl group, in particular a (C₁-C₄)alkyl group and more particularly a methyl,
   a hydroxy group, and
   a (C₁-C₆)alkoxy group, in particular a (C₁-C₄)alkoxy group and more particularly a methoxy group,
      or two R₃, that are borne by the same carbon atom, form with the carbon atom bearing them a (C₃-C₆) cycloalkyl ring, in particular a cyclopropyl ring, unsubstituted or substituted with one or two fluorine atoms; and
R₄ is chosen from:
   a hydrogen atom, and
   a (C₁-C₆)alkyl group, in particular a (C₁-C₄)alkyl group and more particularly a methyl group.

In another embodiment, mention may be made of the compounds of formula (I) for which:
R₁ is a (C₁-C₆)alkyl group, in particular a (C₁-C₄)alkyl group and more particularly a tert-butyl group, substituted with one to three fluorine atoms;
R₂ is a (C₁-C₆)alkyl group, in particular a (C₁-C₄)alkyl group and more particularly a methyl group,
m is 0 or 1;
R₃, when present, is chosen from:
   a hydroxy group, and
   a (C₁-C₆)alkoxy group, in particular a (C₁-C₄)alkoxy group and more particularly a methoxy group,
   preferably R₃ is a hydroxy group; and
R₄ is a hydrogen atom.

The nomenclature of the following compounds (1) to (20) was generated according to the principles of the International Union of Pure and Applied Chemistry, using IUPAC rules for organic compounds.

Among the compounds of formula (I), mention may be made in particular of the following compounds, or a deuterated or tritiated form of the compound of formula (I), or a pharmaceutically acceptable salt thereof:
(1) (S)-2-carbamothioyl-4,4-difluoro-N-(4-methyl-5-(2-(1,1,1-trifluoro-2-methylpropan-2-yl)pyridin-4-yl)thiazol-2-yl)pyrrolidine-1-carboxamide,
(2) (2S,4R)-2-carbamothioyl-4-fluoro-N-(4-methyl-5-(2-(1,1,1-trifluoro-2-methylpropan-2-yl)pyridin-4-yl)thiazol-2-yl)pyrrolidine-1-carboxamide,
(3) (2S,4S)-2-carbamothioyl-4-fluoro-N-(4-methyl-5-(2-(1,1,1-trifluoro-2-methylpropan-2-yl)pyridin-4-yl)thiazol-2-yl)pyrrolidine-1-carboxamide,
(4) (S)-6-carbamothioyl-N-(4-methyl-5-(2-(1,1,1-trifluoro-2-methylpropan-2-yl)pyridin-4-yl)thiazol-2-yl)-1,1-difluoro-5-azaspiro[2,4]heptane-5-carboxamide,
(5) (S)-2-carbamothioyl-N-(4-methyl-5-(2-(1,1,1-trifluoro-2-methylpropan-2-yl)pyridin-4-yl)thiazol-2-yl)pyrrolidine-1-carboxamide,
(6) (S)-2-carbamothioyl-2-methyl-N-(4-methyl-5-(2-(1,1,1-trifluoro-2-methylpropan-2-yl)pyridin-4-yl)thiazol-2-yl)pyrrolidine-1-carboxamide,
(7) (2S,4R)-2-carbamothioyl-4-methoxy-N-(4-methyl-5-(2-(1,1,1-trifluoro-2-methylpropan-2-yl)pyridin-4-yl)thiazol-2-yl)pyrrolidine-1-carboxamide,
(8) (2S,4S)-2-carbamothioyl-4-methoxy-N-(4-methyl-5-(2-(1,1,1-trifluoro-2-methylpropan-2-yl)pyridin-4-yl)thiazol-2-yl)pyrrolidine-1-carboxamide,
(9) (2S,5R)-2-carbamothioyl-5-methyl-N-(4-methyl-5-(2-(1,1,1-trifluoro-2-methylpropan-2-yl)pyridin-4-yl)thiazol-2-yl)pyrrolidine-1-carboxamide,
(10) (2S,5S)-2-carbamothioyl-5-methyl-N-(4-methyl-5-(2-(1,1,1-trifluoro-2-methylpropan-2-yl)pyridin-4-yl)thiazol-2-yl)pyrrolidine-1-carboxamide,
(11) (2S,4S)-2-carbamothioyl-4-methyl-N-(4-methyl-5-(2-(1,1,1-trifluoro-2-methylpropan-2-yl)pyridin-4-yl)thiazol-2-yl)pyrrolidine-1-carboxamide,
(12) (2S,4S)-2-carbamothioyl-N-(4-methyl-5-(2-(1,1,1-trifluoro-2-methylpropan-2-yl)pyridin-4-yl)thiazol-2-yl)-4-(trifluoromethyl)pyrrolidine-1-carboxamide,
(13) (2S,4R)-2-carbamothioyl-4-hydroxy-N-(4-methyl-5-(2-(1,1,1-trifluoro-2-methylpropan-2-yl)pyridin-4-yl)thiazol-2-yl)pyrrolidine-1-carboxamide,
(14) (2S,3S)-2-carbamothioyl-3-hydroxy-N-(4-methyl-5-(2-(1,1,1-trifluoro-2-methylpropan-2-yl)pyridin-4-yl)thiazol-2-yl)pyrrolidine-1-carboxamide,
(15) (2S,3S)-2-carbamothioyl-3-methoxy-N-(4-methyl-5-(2-(1,1,1-trifluoro-2-methylpropan-2-yl)pyridin-4-yl)thiazol-2-yl)pyrrolidine-1-carboxamide,
(16) (S)-6-carbamothioyl-N-(4-methyl-5-(2-(1,1,1-trifluoro-2-methylpropan-2-yl)pyridin-4-yl)thiazol-2-yl)-5-azaspiro[2.4]heptane-5-carboxamide,
(17) (2S,4S)-2-carbamothioyl-4-hydroxy-N-(4-methyl-5-(2-(1,1,1-trifluoro-2-methylpropan-2-yl)pyridin-4-yl)thiazol-2-yl)pyrrolidine-1-carboxamide;
(18) (S)-2-carbamothioyl-N-(4-methyl-5-(2-tert-butyl-pyridin-4-yl)thiazol-2-yl)pyrrolidine-1-carboxamide,
(19) (2S,4S)-2-carbamothioyl-4-fluoro-N-(4-methyl-5-(2-tert-butyl-pyridin-4-yl)thiazol-2-yl)pyrrolidine-1-carboxamide, and
(20) (S)-6-carbamothioyl-N-(4-methyl-5-(2-tert-butyl-pyridin-4-yl)thiazol-2-yl)-5-azaspiro[2.4]heptane-5-carboxamide.

Among the preceding listed compounds, the following compounds may particularly be cited: (5), (7), (8), (13), (14) and (17), or a deuterated or tritiated form of the compound of formula (I), or a pharmaceutical salt thereof.

Among the preceding listed compounds, the following compounds may particularly be cited: (5), (13), (14) and (17), in particular (5), (13) and (14), or a deuterated or tritiated form of the compound of formula (I), or a pharmaceutical salt thereof.

The compounds of the formula (I) can be prepared by the following processes.

Unless otherwise mentioned, R₁, R₂, R₃, R₄ and m are as defined previously.

The compounds of the formula (I) and other related compounds having different substituents are synthesized using techniques and materials described below or otherwise known by the skilled person in the art. In addition, solvents, temperatures, and other reaction conditions presented below may vary as deemed appropriate to the skilled person in the art.

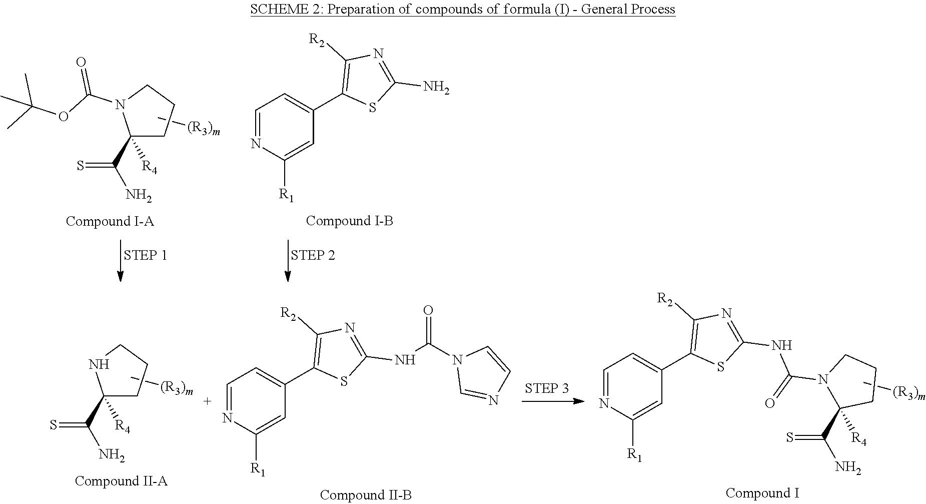

According to SCHEME 1, compound of formula (I) can be obtained in STEP 3 by coupling between compound II-A in which R₄, R₃ and m are as defined above and compound II-B in which R₁ and R₂ are as defined above.

Compound II-A can be obtained from the corresponding compound in which the nitrogen atom is in a protected form, for example from compound I-A having the nitrogen atom protected with a tert-butyl carbamate (Boc) group, by a deprotection STEP 1, for example in an aqueous solution of HCl. More particularly, STEP 1 can be performed by placing compound I-A in a HCl solution with a proper solvent, such as ether or dioxane, and stirred at room temperature (namely at a temperature between 2° and 25° C.) for several hours. The solvent can then be removed in vacuo to obtain compound II-A that is then engaged in the coupling step.

Compound II-B can be obtained from compound I-B in STEP 2 by reaction with 1,1′-carbonyldiimidazole. More particularly, compound I-B can be dissolved in an organic solvent, such as methylene chloride, and 1,1′-carbonyldiimidazole can be added in a molar ratio ranging from 1 to 2, in particular of 1.7. The reaction mixture can be stirred at a temperature ranging between 50° C. and 70° C., for a duration ranging from 12 to 16 hours, and then cooled to room temperature and filtrated to obtain compound II-B as the precipitate.

Compound II-A and compound II-B are then engaged in the coupling STEP 3 at room temperature using triethylamine (TEA) in polar aprotic organic solvent such as N,N-dimethylformamide (DMF) or acetonitrile. More particularly, compounds II-A and II-B can be dissolved in DMF, for example in a molar proportion of 1.3/1, and TEA can then be added at a molar ratio ranging from 2.5 and 3.5, in particular of 3. The reaction mixture can be stirred at room temperature for a duration of between 12 and 16 hours and treated by an aqueous solvent, such as water. The organic phases are extracted, for example by methylene chloride, washed, in particular by brine, and dried, for instance over sodium sulfate. The solvent can then be evaporated, leading to a crude product that can be further purified, in particular by preparative HPLC using a water/acetonitrile gradient, to obtain compound I.

When R₃ is a hydroxy group, R₃ is protected in compounds I-A and II-A before the coupling step, for example with a tert-butyldiphenylsilyl ether (tBDPS) protecting group. After the coupling step between compound II-A with a protected hydroxy group R₃ and compound II-B, deprotection of the hydroxy group, for example using tetra-n-butylammonium fluoride (TBAF) for a tBDPS protecting group, can be performed on the obtained precipitate and before HPLC purification.

Intermediate compound I-A can be prepared by a process as shown in SCHEME 2.

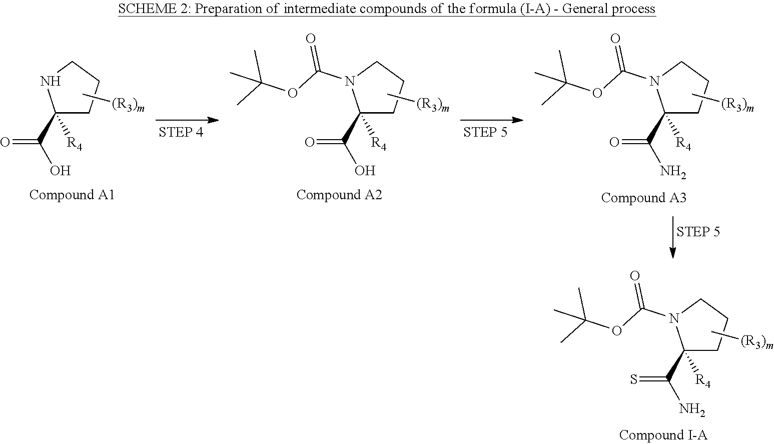

According to SCHEME 2 in which R₃, R₄ and m are as defined above, the nitrogen atom in compound A1 is first protected, for example with a tert-butyl carbamate (Boc) group, in STEP 4. The protection step can be performed by reacting compound A1 with di-tert-butyl dicarbonate in the presence of a base such as triethylamine. For example, compound A1 can be dissolved in an appropriate solvent, such as dichloromethane (DCM). Then, at low temperature, in particular at 0° C., a base is added, such as triethylamine, in a molar ratio ranging from 1 to 2, for example of 1.5, along with di-tert-butyl dicarbonate, in a molar ratio ranging from 1 to 2, for example of 1.2, and an appropriate nucleophilic catalyst, such as 4-dimethylaminopyridine (DMAP), in a molar ratio ranging from 0.01 to 0.5, for example of 0.15. The reaction mixture can then be stirred at low temperature, such as 0° C., for a duration of between 15 and 45 minutes, and then at room temperature for a duration of between 8 and 15 hours. The reaction can then be quenched, for example using sodium bicarbonate (NaHCO₃) in an aqueous solution, and extracted, for example using an appropriate solvent, such as dichloromethane. The organic phase can then be dried, for example over Na₂SO₄ anhydrous, filtered and concentrated, for example in vacuo. The crude product can then be purified, for example using flash chromatography, to obtain compound A2.

Compound A2 can be converted in STEP 5 to compound A3 through suitable conditions to transform the carboxylic acid function into the corresponding amide. For example, STEP 5 can be performed by activation with isobutyl chloroformate in the presence of a base, such as triethylamine, followed by reacting with ammonia, preferably an aqueous solution of ammonia. An extraction with an appropriate organic solvent, for example ethyl acetate, followed by filtration and drying, for example over anhydrous magnesium sulphate, and purification for example by flash chromatography, can give compound A3.

Compound A3 can be converted in STEP 6 to compound A4 by converting the amide function into a thioamide, using for example a Lawesson's reagent.

When R₃ is an hydroxy group, R₃ of compound A2 is protected before performing STEP 5, for example with a tert-butyldiphenylsilyl ether (tBDPS) protecting group. In this case, compound I-A with a protected hydroxy group R₃ is obtained, that can be engaged in the coupling reaction as described above in SCHEME 1.

In a particular embodiment, compound A2 in which R₃ is an alkoxy group can be obtained from a compound A2 in which R₃ is a hydroxy group by converting the hydroxy group into an alkoxy group, for example through a reaction with alkyl iodide in tetrahydrofuran, before performing STEP 5.

Herein is also provided a process for preparing a compound of formula (I) as defined above, comprising a coupling reaction between a compound of formula (II-A) and a compound of formula (II-B)

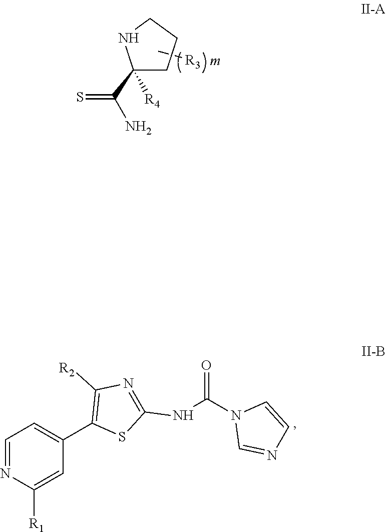

in which R₃, R₄, m, R₂ and R₁ are as defined above, provided that, when R₃ is an hydroxy group, R₃ is protected in compound II-A before the coupling reaction, for example with a tert-butyldiphenylsilyl ether (tBDPS) protecting group; the deprotection being performed after the coupling reaction;
wherein said coupling reaction is preferably performed at room temperature using triethylamine (TEA) in polar aprotic organic solvent such as dimethylformamide (DMF).

Said coupling reaction can be optionally preceded by a step for obtaining compound II-A wherein a compound of formula I-A

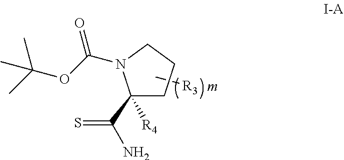

wherein R₃, R₄ and m are as defined above, or R₃ is a protected hydroxy group, for example a hydroxy group protected with a tert-butyldiphenylsilyl ether (tBDPS) protecting group, is converted to a compound II-A by a deprotection step, for example by an aqueous solution of HCl.

Said coupling reaction can be optionally preceded by a step for obtaining compound II-B wherein a compound of formula I-B

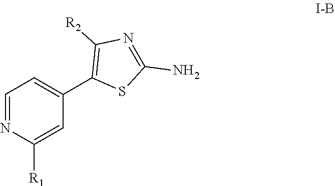

wherein R₁ and R₂ are as defined above, is converted to compound II-B by reaction with 1,1′-carbonyldiimidazole, in particular in an organic solvent, such as methylene chloride.

Herein are further provided the intermediate compounds of formulae (I-A) and (II-A) or, or a deuterated or tritiated form of the compound of formula (I), any of their pharmaceutically acceptable salts:

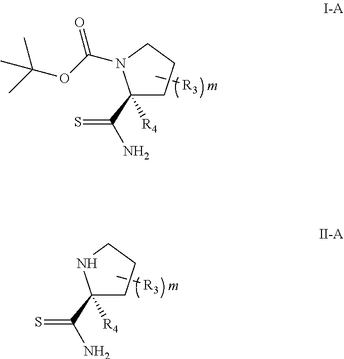

wherein R₃, R₄ and m are as defined above; or R₃ represents a protected hydroxy group, for example a hydroxy group protected with a tert-butyldiphenylsilyl ether (tBDPS) protecting group.

In particular, in intermediate compounds of formula (I-A) or (II-A), m, R₃ and R₄ may have any one of the specific definitions as mentioned above for the compounds of formula (I).

In an embodiment, in the intermediate compounds of formula (I-A) or (II-A):
m is 0, 1 or 2,
each R₃, when present, is independently chosen from:
   a fluorine atom,
   a (C₁-C₆)alkyl group, in particular a (C₁-C₄)alkyl group and more particularly a methyl group, unsubstituted or substituted with one or more halogen atoms,
   a (C₃-C₆) cycloalkyl group unsubstituted or substituted with one or more halogen atoms,
   a hydroxy group, in particular a protected hydroxy group, for example a hydroxy group protected with a tert-butyldiphenylsilyl ether (tBDPS) protecting group;
   a (C₁-C₆)alkoxy group, in particular a (C₁-C₄)alkoxy group and more particularly a methoxy group,
   a —NH₂ group,
      or two R₃, that are borne by the same carbon atom, form with the carbon atom bearing them a (C₃-C₆) cycloalkyl ring, in particular a cyclopropyl ring, unsubstituted or substituted with one or more fluorine atoms, and
R₄ is chosen from:
   a hydrogen atom,
   a (C₁-C₆)alkyl group, in particular a (C₁-C₄)alkyl group and more particularly a methyl group, unsubstituted or substituted with one or more halogen atoms;
      or R₃ and R₄, when they are borne by two adjacent carbon atoms, form with the carbon atoms bearing them a (C₃-C₆) cycloalkyl ring, unsubstituted or substituted with one or more halogen atom.

In an embodiment, in the intermediate compounds of formula (I-A) or (II-A):
m is 0, 1 or 2,
each R₃, when present, is independently chosen from:
   a fluorine atom,
   a (C₁-C₆)alkyl group, in particular a (C₁-C₄)alkyl group and more particularly a methyl,
   a hydroxy group, in particular a protected hydroxy group, for example a hydroxy group protected with a tert-butyldiphenylsilyl ether (tBDPS) protecting group;
   a (C₁-C₆)alkoxy group, in particular a (C₁-C₄)alkoxy group and more particularly a methoxy group,
      or two R₃, that are borne by the same carbon atom, form with the carbon atom bearing them a (C₃-C₆) cycloalkyl ring, in particular a cyclopropyl ring, substituted with one or more fluorine atoms, and
R₄ is chosen from:
   a hydrogen atom, and
   a (C₁-C₆)alkyl group, in particular a (C₁-C₄)alkyl group and more particularly a methyl group.

In another embodiment, in the intermediate compounds of formula (I-A) or (II-A):
m is 0 or 1;
R₃, when present, is chosen from:
   a hydroxy group, and
   a (C₁-C₆)alkoxy group, in particular a (C₁-C₄)alkoxy group and more particularly a methoxy group,
   preferably R₃ is a hydroxy group; and
R₄ is a hydrogen atom.

Among the intermediate compounds of formula (I-A), mention may be made in particular of the following compounds, or a deuterated or tritiated form of the compound of formula (I), or a pharmaceutically acceptable salt thereof:
(21) tert-butyl(S)-2-carbamothioyl-4,4-difluoropyrrolidine-1-carboxylate;
(22) tert-butyl(2S,4R)-2-carbamothioyl-4-fluoropyrrolidine-1-carboxylate,
(23) tert-butyl(2S,4S)-2-carbamothioyl-4-fluoropyrrolidine-1-carboxylate,
(24) tert-butyl (6S)-6-carbamothioyl-1,1-difluoro-5-azaspiro[2.4]heptane-5-carboxylate,
(25) tert-butyl(S)-2-carbamothioylpyrrolidine-1-carboxylate,
(26) tert-butyl(S)-2-carbamothioyl-2-methylpyrrolidine-1-carboxylate,
(27) tert-butyl(2S,4R)-2-carbamothioyl-4-methoxypyrrolidine-1-carboxylate,
(28) tert-butyl(2S,4S)-2-carbamothioyl-4-methoxypyrrolidine-1-carboxylate,
(29) tert-butyl(2S,5R)-2-carbamothioyl-5-methylpyrrolidine-1-carboxylate,
(30) tert-butyl(2S,5S)-2-carbamothioyl-5-methylpyrrolidine-1-carboxylate,
(31) tert-butyl(2S,4S)-2-carbamothioyl-4-methylpyrrolidine-1-carboxylate,
(32) tert-butyl(2S,4S)-2-carbamothioyl-4-(trifluoromethyl)pyrrolidine-1-carboxylate,
(33) tert-butyl(2S,4R)-4-((tert-butyldiphenylsilyl)oxy)-2-carbamothioylpyrrolidine-1-carboxylate,
(34) tert-butyl(2S,3S)-3-((tert-butyldiphenylsilyl)oxy)-2-carbamothioylpyrrolidine-1-carboxylate,
(35) tert-butyl(2S,3S)-2-carbamothioyl-3-methoxypyrrolidine-1-carboxylate,
(36) tert-butyl(S)-6-carbamothioyl-5-azaspiro[2.4]heptane-5-carboxylate, and
(37) tert-butyl(2S,4S)-4-((tert-butyldiphenylsilyl)oxy)-2-carbamothioylpyrrolidine-1-carboxylate.

More particularly, a compound according to the invention may be selected from the group consisting of the following compounds, or a deuterated or tritiated form of the compound of formula (I), or a pharmaceutically acceptable salt thereof:
(21) tert-butyl(S)-2-carbamothioyl-4,4-difluoropyrrolidine-1-carboxylate;
(23) tert-butyl(2S,4S)-2-carbamothioyl-4-fluoropyrrolidine-1-carboxylate,
(24) tert-butyl (6S)-6-carbamothioyl-1,1-difluoro-5-azaspiro[2.4]heptane-5-carboxylate,
(26) tert-butyl(S)-2-carbamothioyl-2-methylpyrrolidine-1-carboxylate,
(28) tert-butyl(2S,4S)-2-carbamothioyl-4-methoxypyrrolidine-1-carboxylate,
(29) tert-butyl(2S,5R)-2-carbamothioyl-5-methylpyrrolidine-1-carboxylate,
(30) tert-butyl(2S,5S)-2-carbamothioyl-5-methylpyrrolidine-1-carboxylate,
(31) tert-butyl(2S,4S)-2-carbamothioyl-4-methylpyrrolidine-1-carboxylate,
(32) tert-butyl(2S,4S)-2-carbamothioyl-4-(trifluoromethyl)pyrrolidine-1-carboxylate,
(34) tert-butyl(2S,3S)-3-((tert-butyldiphenylsilyl)oxy)-2-carbamothioylpyrrolidine-1-carboxylate,
(35) tert-butyl(2S,3S)-2-carbamothioyl-3-methoxypyrrolidine-1-carboxylate,
(36) tert-butyl(S)-6-carbamothioyl-5-azaspiro[2.4]heptane-5-carboxylate, and
(37) tert-butyl(2S,4S)-4-((tert-butyldiphenylsilyl)oxy)-2-carbamothioylpyrrolidine-1-carboxylate.

As for the intermediate compounds of formula (II-B), R₁ and R₂ may have any one of the specific definitions as mentioned above for the compounds of formula (I).

In an embodiment, in the intermediate compounds of formula (II-B):
R₁ is a (C₁-C₆)alkyl group, in particular a (C₁-C₄)alkyl group and more particularly a tert-butyl group, unsubstituted or substituted with one or more fluorine atoms,
R₂ is chosen from:
   a hydrogen atom, and
   a (C₁-C₆)alkyl group, in particular a (C₁-C₄)alkyl group and more particularly a methyl group.

In another embodiment, in the intermediate compounds of formula (III-B):
R₁ is a (C₁-C₆)alkyl group, in particular a (C₁-C₄)alkyl group and more particularly a tert-butyl group, unsubstituted or substituted with one to three fluorine atoms, and
R₂ is a (C₁-C₆)alkyl group, in particular a (C₁-C₄)alkyl group and more particularly a methyl group.

Intermediate compounds of formula (II-B) may be commercially available. One may mention for example 4-Methyl-5-[2-(2,2,2-trifluoro-1,1-dimethylethyl)-4-pyridinyl]-2-thiazolamine (CAS 1357476-69-7) sold by AURUM pharmatech, 5-[2-(2,2,2-Trifluoro-1,1-dimethylethyl)-4-pyridinyl]-2-thiazolamine (CAS 1395492-61-1) sold by Carbosynth and 5-[2-(1,1-Dimethylethyl)-4-pyridinyl]-2-thiazolamine (CAS 1395492-83-7) sold by Matrix Scientific.

The following tables 1a and 1b comprise respectively specific compounds of formula (I) (basic formula and structure) in accordance with the present disclosure as well as their characterization (specific optical rotation, ¹H NMR and high-resolution electrospray ionization mass spectrometry HRMS-ESI).

Optical rotations (αD) were measured on a Perkin Elmer polarimeter (model 341) at 20° C.

¹H NMR and ¹³C NMR spectra were recorded on Bruker Avance II 500 spectrometer, at 500 MHz (H value) or 125 MHz (C value) with the chemical shifts (8 in ppm) in the solvent dimethyl sulfoxide-d6 (d6-DMSO) referenced at 2.5 ppm at a temperature of 300 K. Coupling constants (J) are reported in Hertz.

High Resolution mass spectra were recorded on a ThermoFischer Exactive Orbitrap spectrometer.

TABLE 1a
Compounds (1) to (20).

| Cpd | Basic | | |
|---|---|---|---|
| Nº | formula | Structure | Name |
| 1 | C₁₉H₂₀F₅N₅O_{S}2 | 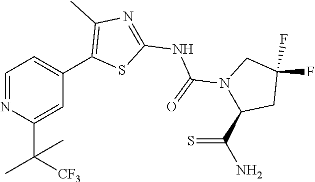 | (S)-2-carbamothioyl-4,4- difluoro-N-(4-methyl-5-(2- (1,1,1-trifluoro-2-methylpropan- 2-yl)pyridin-4-yl)thiazol-2- yl)pyrrolidine-1-carboxamide |
| 2 | C₁₉H₂₁F₄N₅OS₂ | 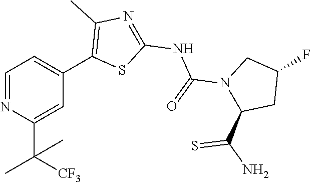 [SMILES: Cc1nc(NC(=O)N2C[C@H](F)C[C@H]2C(N)=S)sc1-c1ccnc(C(C)(C)C(F)(F)F)c1] | (2S,4R)-2-carbamothioyl-4- fluoro-N-(4-methyl-5-(2-(1,1,1- trifluoro-2-methylpropan-2- yl)pyridin-4-yl)thiazol-2- yl)pyrrolidine-1-carboxamide |
| 3 | C₁₉H₂₁F₄N₅OS₂ | 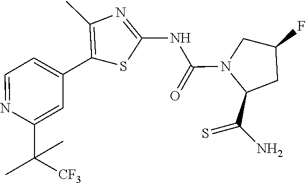 [SMILES: Cc1nc(NC(=O)N2C[C@@H](F)C[C@H]2C(N)=S)sc1-c1ccnc(C(C)(C)C(F)(F)F)c1] | (2S,4S)-2-carbamothioyl-4- fluoro-N-(4-methyl-5-(2-(1,1,1- trifluoro-2-methylpropan-2- yl)pyridin-4-yl)thiazol-2- yl)pyrrolidine-1-carboxamide |
| 4 | C₂₁H₂₂F₅N₅OS₂ | 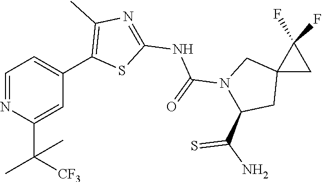 | (S)-6-carbamothioyl-N-(4- methyl-5-(2-(1,1,1-trifluoro-2- methylpropan-2-yl)pyridin-4- yl)thiazol-2-yl)-1,1-difluoro-5- azaspiro[2,4]heptane-5- carboxamide |
| 5 | C₁₉H₂₂F₃N₅OS₂ | 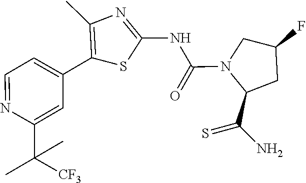 [SMILES: Cc1nc(NC(=O)N2C[C@@H](F)C[C@H]2C(N)=S)sc1-c1ccnc(C(C)(C)C(F)(F)F)c1] | (S)-2-carbamothioyl-N-(4- methyl-5-(2-(1,1,1-trifluoro-2- methylpropan-2-yl)pyridin-4- yl)thiazol-2-yl)pyrrolidine-1- carboxamide |
| 6 | C₂₀H₂₄F₃N₅OS₂ | 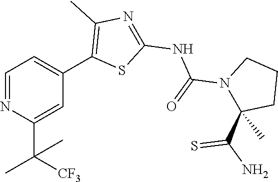 [SMILES: Cc1nc(NC(=O)N2CCC[C@@]2(C)C(N)=S)sc1-c1ccnc(C(C)(C)C(F)(F)F)c1] | (S)-2-carbamothioyl-2-methyl- N-(4-methyl-5-(2-(1,1,1- trifluoro-2-methylpropan-2- yl)pyridin-4-yl)thiazol-2- yl)pyrrolidine-1-carboxamide |
| 7 | C₂₀H₂₄F₃N₅O₂S₂ | 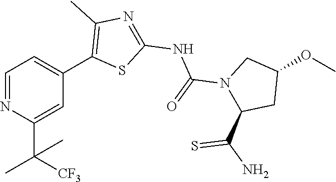 [SMILES: CO[C@@H]1C[C@@H](C(N)=S)N(C(=O)Nc2nc(C)c(-c3ccnc(C(C)(C)C(F)(F)F)c3)s2)C1] | (2S,4R)-2-carbamothioyl-4- methoxy-N-(4-methyl-5-(2- (1,1,1-trifluoro-2-methylpropan- 2-yl)pyridin-4-yl)thiazol-2- yl)pyrrolidine-1-carboxamide |
| 8 | C₂₀H₂₄F₃N₅O₂S₂ | 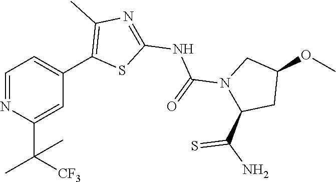 [SMILES: CO[C@H]1C[C@@H](C(N)=S)N(C(=O)Nc2nc(C)c(-c3ccnc(C(C)(C)C(F)(F)F)c3)s2)C1] | (2S,4S)-2-carbamothioyl-4- methoxy-N-(4-methyl-5-(2- (1,1,1-trifluoro-2-methylpropan- 2-yl)pyridin-4-yl)thiazol-2- yl)pyrrolidine-1-carboxamide |
| 9 | C₂₀H₂₄F₃N₅OS₂ | 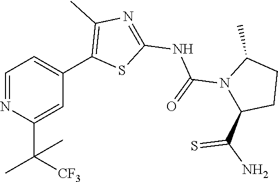 [SMILES: Cc1nc(NC(=O)N2[C@H](C)CC[C@H]2C(N)=S)sc1-c1ccnc(C(C)(C)C(F)(F)F)c1] | (2S,5R)-2-carbamothioyl-5- methyl-N-(4-methyl-5-(2-(1,1,1- trifluoro-2-methylpropan-2- yl)pyridin-4-yl)thiazol-2- yl)pyrrolidine-1-carboxamide |
| 10 | C₂₀H₂₄F₃N₅OS₂ | 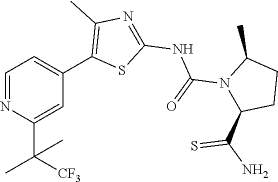 [SMILES: Cc1nc(NC(=O)N2[C@@H](C)CC[C@H]2C(N)=S)sc1-c1ccnc(C(C)(C)C(F)(F)F)c1] | (2S,5S)-2-carbamothioyl-5- methyl-N-(4-methyl-5-(2-(1,1,1- trifluoro-2-methylpropan-2- yl)pyridin-4-yl)thiazol-2- yl)pyrrolidine-1-carboxamide |
| 11 | C₂₀H₂₄F₃N₅OS₂ | 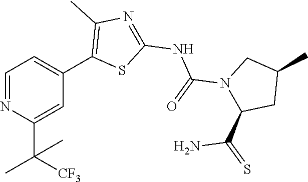 [SMILES: Cc1nc(NC(=O)N2C[C@@H](C)C[C@H]2C(N)=S)sc1-c1ccnc(C(C)(C)C(F)(F)F)c1] | (2S,4S)-2-carbamothioyl-4- methyl-N-(4-methyl-5-(2-(1,1,1- trifluoro-2-methylpropan-2- yl)pyridin-4-yl)thiazol-2- yl)pyrrolidine-1-carboxamide |
| 12 | C₂₀H₂₁F₆N₅OS₂ | 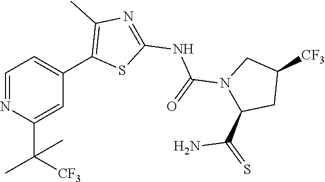 [SMILES: Cc1nc(NC(=O)N2C[C@@H](C(F)(F)F)C[C@H]2C(N)=S)sc1-c1ccnc(C(C)(C)C(F)(F)F)c1] | (2S,4S)-2-carbamothioyl-N-(4- methyl-5-(2-(1,1,1-trifluoro-2- methylpropan-2-yl)pyridin-4- yl)thiazol-2-yl)-4- (trifluoromethyl)pyrrolidine-1- carboxamide |
| 13 | C₁₉H₂₂F₃N₅O₂S₂ | 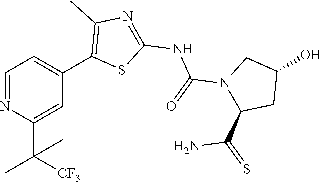 [SMILES: Cc1nc(NC(=O)N2C[C@H](O)C[C@H]2C(N)=S)sc1-c1ccnc(C(C)(C)C(F)(F)F)c1] | (2S,4R)-2-carbamothioyl-4- hydroxy-N-(4-methyl-5-(2- (1,1,1-trifluoro-2-methylpropan- 2-yl)pyridin-4-yl)thiazol-2- yl)pyrrolidine-1-carboxamide |
| 14 | C₁₉H₂₂F₃N₅O₂S₂ | 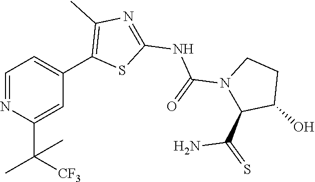 [SMILES: Cc1nc(NC(=O)N2CC[C@H](O)[C@H]2C(N)=S)sc1-c1ccnc(C(C)(C)C(F)(F)F)c1] | (2S,3S)-2-carbamothioyl-3- hydroxy-N-(4-methyl-5-(2- (1,1,1-trifluoro-2-methylpropan- 2-yl)pyridin-4-yl)thiazol-2- yl)pyrrolidine-1-carboxamide |
| 15 | C₂₀H₂₄F₃N₅O₂S₂ | 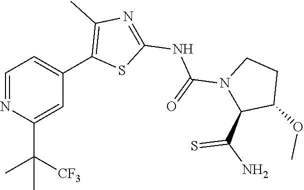 [SMILES: CO[C@H]1CCN(C(=O)Nc2nc(C)c(-c3ccnc(C(C)(C)C(F)(F)F)c3)s2)[C@@H]1C(N)=S] | (2S,3S)-2-carbamothioyl-3- methoxy-N-(4-methyl-5-(2- (1,1,1-trifluoro-2-methylpropan- 2-yl)pyridin-4-yl)thiazol-2- yl)pyrrolidine-1-carboxamide |
| 16 | C₂₁H₂₄F₃N₅OS₂ | 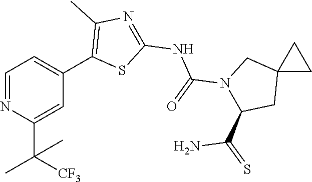 [SMILES: Cc1nc(NC(=O)N2CC3(CC3)C[C@H]2C(N)=S)sc1-c1ccnc(C(C)(C)C(F)(F)F)c1] | (S)-6-carbamothioyl-N-(4- methyl-5-(2-(1,1,1-trifluoro-2- methylpropan-2-yl)pyridin-4- yl)thiazol-2-yl)-5- azaspiro[2.4]heptane-5- carboxamide |
| 17 | C₁₉H₂₂F₃N₅O₂S₂ | 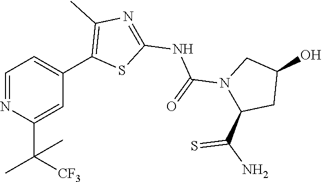 [SMILES: Cc1nc(NC(=O)N2C[C@@H](O)C[C@H]2C(N)=S)sc1-c1ccnc(C(C)(C)C(F)(F)F)c1] | (2S,4S)-2-carbamothioyl-4- hydroxy-N-(4-methyl-5-(2- (1,1,1-trifluoro-2-methylpropan- 2-yl)pyridin-4-yl)thiazol-2- yl)pyrrolidine-1-carboxamide |
| 18 | C₁₉H₂₅N₅OS₂ | 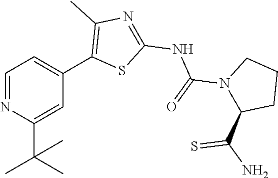 [SMILES: Cc1nc(NC(=O)N2CCC[C@H]2C(N)=S)sc1-c1ccnc(C(C)(C)C)c1] | (S)-2-carbamothioyl-N-(4- methyl-5-(2-tert-butyl-pyridin-4- yl)thiazol-2-yl)pyrrolidine-1- carboxamide |
| 19 | C₁₉H₂₅FN₅OS₂ | 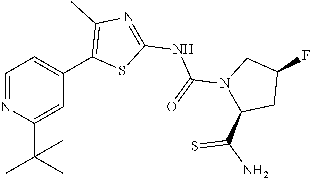 [SMILES: Cc1nc(NC(=O)N2C[C@@H](F)C[C@H]2C(N)=S)sc1-c1ccnc(C(C)(C)C)c1] | (2S,4S)-2-carbamothioyl-4- fluoro-N-(4-methyl-5-(2-tert- butyl-pyridin-4-yl)thiazol-2- yl)pyrrolidine-1-carboxamide |
| 20 | C₂₁H₂₇N₅OS₂ | 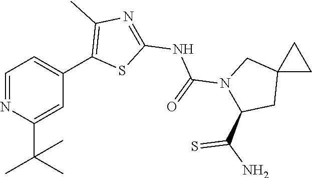 [SMILES: Cc1nc(NC(=O)N2CC3(CC3)C[C@H]2C(N)=S)sc1-c1ccnc(C(C)(C)C)c1] | (S)-6-carbamothioyl-N-(4- methyl-5-(2-tert-butyl-pyridin-4- yl)thiazol-2-yl)-5- azaspiro[2.4]heptane-5- carboxamide |

TABLE 1b
Characterization of compounds (1) to (20)

| Cpd | | |
|---|---|---|
| No | Yield | Characterization |
| 1 | 72% | [α]_{D} +8.4 (c 0.095, CHCl₃). ¹H NMR (500 MHz, DMSO) δ 9.81 (s, 1H), 9.40 (s, |
| | | 1H), 8.61 (d, J = 5.2 Hz, 1H), 7.55 (s, 1H), 7.42 (dd, J = 5.1, 1.3 Hz, 1H), 4.89 |
| | | (s, 1H), 4.07 (d, J = 10.9 Hz, 2H), 3.07-2.89 (m, 1H), 2.52 (d, J = 3.2 Hz, 1H), |
| | | 2.44-2.33 (m, 3H), 1.62 (s, 7H). HRMS ESI⁺ calculated 494.1102 for |
| | | C₁₉H₂₁F₅N₅OS₂, found 494.1101 |
| 2 | 58% | [α]_{D} −63.5 (c 0.085, CHCl₃). ¹H NMR (500 MHz, DMSO) δ 11.22 (s, 1H), 9.65 |
| | | (s, 1H), 9.40 (s, 1H), 8.60 (d, J = 5.1 Hz, 1H), 7.56 (s, 1H), 7.42 (dd, J = 5.1, 1.5 |
| | | Hz, 1H), 5.40 (d, J = 53.1 Hz, 1H), 4.88-4.65 (m, 1H), 4.00 (dt, J = 32.9, 16.3 |
| | | Hz, 1H), 3.75 (dd, J = 38.0, 11.7 Hz, 1H), 2.62-2.51 (m, 1H), 2.41 (s, 3H), 2.20- |
| | | 2.02 (m, 1H), 1.61 (s, 6H). |
| | | HRMS ESI⁺ calculated 476.1196 for C₁₉H₂₂F₄N₅OS₂, found 476.1195 |
| 3 | 61% | [α]_{D} +74 (c 0.1, CHCl₃). ¹H NMR (500 MHz, DMSO) δ 11.10 (s, 1H), 9.70 (s, |
| | | 1H), 9.20 (s, 1H), 8.61 (d, J = 5.1 Hz, 1H), 7.55 (s, 1H), 7.42 (dd, J = 5.1, 1.2 |
| | | Hz, 1H), 5.31 (d, J = 53.1 Hz, 1H), 4.79 (d, J = 7.3 Hz, 1H), 3.97 (s, 1H), 3.78 |
| | | (ddd, J = 36.8, 12.5, 3.9 Hz, 1H), 2.68-2.51 (m, 2H), 2.42 (s, 3H), 1.61 (s, 6H). |
| | | HRMS ESI⁺ calculated 476.1196 for C₁₉H₂₂F₄N₅OS₂, found 476.1192 |
| 4 | 59% | [α]_{D} −93.3 (c 0.075, CHCl₃). ¹H NMR (500 MHz, DMSO) δ 11.13 (s, 1H), 9.71 |
| | | (s, 1H), 9.32 (s, 1H), 8.61 (d, J = 5.1 Hz, 1H), 7.55 (s, 1H), 7.42 (d, J = 4.2 Hz, |
| | | 1H), 4.81 (m, 1H), 3.96-3.78 (m, 1H), 3.78-3.61 (m, 1H), 2.63 (dt, J = 17.0, |
| | | 7.6 Hz, 1H), 2.40 (s, 3H), 2.36 (dt, J = 3.5, 1.8 Hz, 1H), 1.99 (d, J = 12.3 Hz, |
| | | 1H), 1.61 (s, 6H), 1.55 (m, 1H). |
| | | HRMS ESI⁺ calculated 520.1259 for C₂₁H₂₃F₅N₅OS₂, found 520.1253 |
| 5 | 73% | [α]_{D} −144 (c 0.125, CHCl₃). ¹H NMR (500 MHz, DMSO) δ 10.96 (s, 1H), 9.60 |
| | | (s, 1H), 9.19 (s, 1H), 8.60 (d, J = 5.2 Hz, 1H), 7.55 (s, 1H), 7.41 (dd, J = 5.2, 1.4 |
| | | Hz, 1H), 4.67 (s, 1H), 3.71 (s, 1H), 3.54-3.43 (m, 1H), 2.41 (s, 3H), 2.27-2.15 |
| | | (m, 1H), 1.89 (m, 3H), 1.61 (s, 6H). HRMS ESI⁺ calculated 458.1291 for |
| | | C₁₉H₂₃F₃N₅OS₂, found 458.1270 |
| 6 | 58% | [α]_{D} +26 (c 0.1, CHCl₃). ¹H NMR (500 MHz, DMSO) δ 10.21 (s, 1H), 8.67 (dd, |
| | | J = 5.1, 0.4 Hz, 1H), 7.64 (s, 1H), 7.50 (dd, J = 5.1, 1.6 Hz, 1H), 3.63 (dt, J = |
| | | 11.0, 8.3 Hz, 1H), 3.38-3.29 (m, 1H), 2.49 (s, 3H), 2.47-2.30 (m, 1H), 2.22- |
| | | 2.02 (m, 2H), 2.00-1.85 (m, 2H), 1.63 (s, 6H), 1.46 (s, 3H). HRMS ESI⁺ |
| | | calculated 472.1447 for C₂₀H₂₅F₃N₅OS₂, found 472.1818 |
| 7 | 55% | [α]_{D} −37 (c 0.075, CHCl₃). ¹H NMR (500 MHz, DMSO) δ 11.07 (s, 1H), 9.59 (s, |
| | | 1H), 9.31 (s, 1H), 8.60 (d, J = 5.1 Hz, 1H), 7.55 (s, 1H), 7.42 (dd, J = 5.1, 1.3 |
| | | Hz, 1H), 4.67 (d, J = 6.0 Hz, 1H), 4.04 (s, 1H), 3.76 (d, J = 11.0 Hz, 1H), 3.67 |
| | | (d, J = 8.0 Hz, 1H), 3.22 (s, 3H), 2.41 (s, 3H), 2.33 (s, 1H), 1.98 (s, 1H), 1.61 (s, |
| | | 6H). HRMS ESI⁺ calculated 488.1396 for C₂₀H₂₅F₃N₅O₂S₂, found 488.1392 |
| 8 | 65% | [α]_{D} +55 (c 0.1, CHCl₃). ¹H NMR (500 MHz, DMSO) δ 11.00 (s, 1H), 9.59 (s, |
| | | 1H), 9.07 (s, 1H), 8.60 (d, J = 5.1 Hz, 1H), 7.55 (s, 1H), 7.41 (dd, J = 5.1, 1.3 |
| | | Hz, 1H), 4.66 (s, 1H), 3.97 (s, 1H), 3.74 (dd, J = 11.0, 5.2 Hz, 1H), 3.67 (t, J = |
| | | 8.4 Hz, 1H), 3.18 (s, 3H), 2.44-2.30 (m, 1H), 2.42 (s, 3H), 2.26 (s, 1H), 1.61 (s, |
| | | 6H). HRMS ESI⁺ calculated 488.1396 for C₂₀H₂₅F₃N₅O₂S₂, found 488.1391 |
| 9 | 51% | [α]_{D} −92.8 (c 0.07, CHCl₃). ¹H NMR (500 MHz, DMSO) δ 10.16 (s, 1H), 8.60 |
| | | (d, J = 5.2 Hz, 1H), 7.55 (s, 1H), 7.42 (dd, J = 5.1, 1.4 Hz, 1H), 4.61-4.46 (m, |
| | | 1H), 3.95 (dd, J = 14.2, 6.9 Hz, 1H), 2.39 (m, 4H), 2.34-2.15 (m, 2H), 1.88- |
| | | 1.74 (m, 2H), 1.74-1.66 (m, 1H), 1.62 (s, 6H), 1.13 (d, J = 5.9 Hz, 3H). HRMS |
| | | ESI⁺ calculated 472.1447 for C₂₀H₂₅F₃N₅OS₂, found 472.1443 |
| 10 | 66% | [α]_{D} −37 (c 0.135, CHCl₃). ¹H NMR (500 MHz, DMSO) δ 9.60 (s, 1H), 9.02 (s, |
| | | 1H), 8.60 (d, J = 5.2 Hz, 1H), 7.54 (s, 1H), 7.41 (dd, J = 5.2, 1.5 Hz, 1H), 4.63 |
| | | (d, J = 6.8 Hz, 1H), 4.22 (s, 1H), 2.40 (s, 3H), 2.29-2.13 (m, 1H), 2.13-1.92 |
| | | (m, 2H), 1.66-1.55 (m, 8H), 1.30 (d, J = 6.3 Hz, 3H). HRMS ESI⁺ calculated |
| | | 472.1447 for C₂₀H₂₅F₃N₅OS₂, found 472.1434 |
| 11 | 60% | [α]_{D} −32.7 (c 0.165, MeOH). ¹H NMR (500 MHz, DMSO) δ 10.96 (s, 1H), |
| | | 9.52 (s, 1H), 9.24 (s, 1H), 8.59 (d, J = 5.2 Hz, 2H), 7.54 (s, 2H), 7.41 (dd, J = |
| | | 5.1, 1.3 Hz, 2H), 4.63 (s, 1H), 3.87 (t, J = 8.0 Hz, 2H), 3.10 (t, J = 9.9 Hz, 1H), |
| | | 2.41 (s, 6H), 2.22 (d, J = 6.2 Hz, 2H), 1.61 (s, 10H), 1.48 (dd, J = 20.6, 11.2 |
| | | Hz, 2H), 1.00 (d, J = 6.5 Hz, 5H). HRMS ESI⁺ calculated 472.1447 for |
| | | C₂₀H₂₅F₃N₅OS₂, found 472.1447 |
| 12 | 49% | [α]_{D} 0 (c 0.11, CHCl₃). ¹H NMR (500 MHz, DMSO) δ 11.20 (s, 1H), 9.66 (s, |
| | | 1H), 9.37 (s, 1H), 8.60 (d, J = 5.1 Hz, 1H), 7.55 (s, 1H), 7.42 (dd, J = 5.1, 1.2 |
| | | Hz, 1H), 4.77 (s, 1H), 4.09 (t, J = 8.2 Hz, 1H), 3.59 (s, 1H), 3.39 (m, 1H), 2.69- |
| | | 2.55 (m, 1H), 2.41 (s, 3H), 2.02-1.87 (m, 1H), 1.61 (s, 6H). |
| | | HRMS ESI⁺ calculated 526.1164 for C₂₀H₂₂F₆N₅OS₂, found 526.1161 |
| 13 | 29% | [α]_{D} −61 (c 0.1, CHCl₃). ¹H NMR (500 MHz, DMSO) δ 11.04 (s, 1H), 9.54 (s, |
| | | 1H), 9.28 (s, 1H), 8.60 (d, J = 5.2 Hz, 1H), 7.55 (s, 1H), 7.42 (d, J = 4.2 Hz, 1H), |
| | | 5.14 (s, 1H), 4.72 (s, 1H), 4.33 (s, 1H), 3.67 (d, J = 7.4 Hz, 1H), 3.53 (d, J = 10.5 |
| | | Hz, 1H), 2.41 (s, 3H), 2.17 (m, 1H), 1.93 (m, 1H), 1.61 (s, 6H).. HRMS ESI⁻ |
| | | calculated 472.1094 for C₁₉H₂₁F₃N₅O₂S₂, found 472.1101 |
| 14 | 36% | [α]_{D} 0 (c 0.1, CHCl₃). ¹H NMR (500 MHz, DMSO) δ 11.02 (s, 1H), 9.70 (s, |
| | | 1H), 9.21 (s, 1H), 8.60 (d, J = 5.1 Hz, 1H), 7.55 (s, 1H), 7.42 (d, J = 4.3 Hz, |
| | | 1H), 5.46 (s, 1H), 4.52 (s, 1H), 4.28 (s, 1H), 3.79 (s, 1H), 3.58 (dd, J = 17.0, |
| | | 9.7 Hz, 1H), 2.42 (s, 3H), 1.96 (m, 1H), 1.80 (s, 1H), 1.61 (s, 6H). |
| | | HRMS ESI⁻ calculated 472.1094 for C₁₉H₂₁F₃N₅O₂S₂, found 472.1100 |
| 15 | 76% | [α]_{D} −128.6(c 0.105, CHCl₃). ¹H NMR (500 MHz, DMSO) δ 11.07 (s, 1H), |
| | | 9.79 (s, 1H), 9.34 (s, 1H), 8.61 (d, J = 5.2 Hz, 1H), 7.56 (s, 1H), 7.43 (d, J = |
| | | 4.4 Hz, 1H), 4.69 (m, 1H), 3.93 (m, 1H), 3.84 (m, 1H), 3.48 (q, J = 9.0 Hz, |
| | | 1H), 3.33 (s, 3H), 2.43 (s, 3H), 2.01 (m, 2H), 1.62 (s, 6H). HRMS ESI⁺ |
| | | calculated 488.1396 for C₂₀H₂₄F₃N₅O₂S₂, found 488.1394 |
| 16 | 46% | [α]_{D} −9.1 (c 0.11, CHCl₃). ¹H NMR (500 MHz, DMSO) δ 10.93 (s, 1H), 9.66 (s, |
| | | 1H), 9.21 (s, 1H), 8.60 (d, J = 5.1 Hz, 1H), 7.55 (s, 1H), 7.41 (dd, J = 5.2, 1.4 |
| | | Hz, 1H), 4.78 (m, 1H), 3.54 (m, 2H), 2.39 (m, 4H), 1.88-1.77 (m, 1H), 1.61 |
| | | (s, 6H), 0.77-0.31 (m, 4H). HRMS ESI⁺ calculated 484.1447 for |
| | | C₂₁H₂₅F₃N₅OS₂, found 484.1445 |
| 17 | 40% | [α]_{D} −54.8 (c 0.135, CHCl₃). ¹H NMR (500 MHz, DMSO) δ 10.98 (s, 1H), 9.57 |
| | | (s, 1H), 9.12 (s, 1H), 8.63-8.53 (m, 1H), 7.54 (s, 1H), 7.41 (dd, J = 5.2, 1.6 |
| | | Hz, 1H), 5.10 (s, 1H), 4.64 (m, 1H), 4.24 (s, 1H), 3.75 (dd, J = 10.5, 5.6 Hz, |
| | | 1H), 3.55-3.43 (m, 1H), 2.41 (m, 4H), 2.03-1.90 (m, 1H), 1.61 (s, 6H). |
| | | HRMS ESI⁻ calculated 472.1094 for C₁₉H₂₁F₃N₅O₂S₂, found 472.1103 |
| 18 | 53% | [α]_{D} −36 (c 0.1, CHCl₃). ¹H NMR (500 MHz, DMSO) δ 10.94 (s, 1H), 9.60 (s, |
| | | 1H), 9.19 (s, 1H), 8.51 (m, 1H), 7.36 (d, J = 0.8 Hz, 1H), 7.23 (dd, J = 5.2, 1.6 |
| | | Hz, 1H), 4.67 (s, 1H), 3.70 (s, 1H), 3.48 (m, 1H), 2.41 (s, 3H), 2.25-2.15 (m, |
| | | 1H), 1.89 (m, 3H), 1.34 (s, 9H). HRMS ESI⁺ calcd 404.1573 for C₁₉H₂₆N₅OS₂, |
| | | found 404.1571 |
| 19 | 70% | [α]_{D} +32 (c 0.155, MeOH). ¹H NMR (500 MHz, DMSO) δ 11.05 (s, 1H), 9.69 |
| | | (s, 1H), 9.19 (s, 1H), 8.52 (dd, J = 5.1, 0.5 Hz, 1H), 7.36 (s, 1H), 7.24 (dd, J = |
| | | 5.1, 1.5 Hz, 1H), 5.25 (dd, J = 55.3, 28.5 Hz, 1H), 4.78 (d, J = 8.6 Hz, 1H), |
| | | 3.95 (m, 1H), 3.77 (ddd, J = 36.8, 12.5, 4.0 Hz, 1H), 2.69-2.50 (m, 2H), 2.37 |
| | | (s, 3H), 1.33 (s, 9H). HRMS ESI⁺ calcd 422.1479 for C₁₉H₂₅FN₅OS₂, found |
| | | 422.1490 |
| 20 | 68% | [α]_{D} +13.6 (c 0.22, MeOH). ¹H NMR (500 MHz, DMSO) δ 10.89 (s, 1H), 9.66 |
| | | (s, 1H), 9.21 (s, 1H), 8.52 (d, J = 5.2 Hz, 1H), 7.35 (m, 1H), 7.24 (dd, J = 5.1, |
| | | 1.5 Hz, 1H), 4.74 (m, 1H), 3.66-3.44 (m, 2H), 2.39 (m, 4H), 1.88-1.77 (m, |
| | | 1H), 1.35 (s, 9H), 0.68-0.39 (m, 4H). HRMS ESI⁺ calcd 430.1730 for |
| | | C₂₁H₂₈N₅OS₂, found 430.1720 |

The following tables 2a and 2b comprise respectively specific intermediate compounds of formula (I-A) (basic formula and structure) in accordance with the present disclosure as well as their characterization (specific optical rotation, ¹H NMR, ¹³C NMR and high-resolution electrospray ionization mass spectrometry HRMS-ESI).

Optical rotations (αD) were measured on a Perkin Elmer polarimeter (model 341) at 20° C.

¹H NMR and ¹³C NMR spectra were recorded on Bruker Avance II 500 spectrometer, at 500 MHz (H value) or 125 MHz (C value) with the chemical shifts (8 in ppm) in the solvent dimethyl sulfoxide-d6 (d6-DMSO) referenced at 2.5 ppm at a temperature of 300 K. Coupling constants (J) are reported in Hertz.

High Resolution mass spectra were recorded on a ThermoFischer Exactive Orbitrap spectrometer.

TABLE 2a
Intermediate compounds (21) to (37).

| Cpd | | | |
|---|---|---|---|
| Nº | Basic formula | Structure | Name |
| 21 | C₁₀H₁₆F₂N₂O₂S | 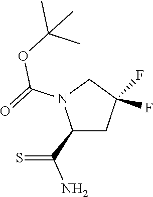 | tert-butyl (S)-2-carbamothioyl- 4,4-difluoropyrrolidine-1- carboxylate |
| 22 | C₁₀H₁₇FN₂O₂S | 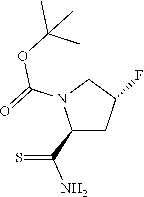 [SMILES: CC(C)(C)OC(=O)N1C[C@H](F)C[C@H]1C(N)=S] | tert-butyl (2S,4R)-2- carbamothioyl-4- fluoropyrrolidine-1-carboxylate |
| 23 | C₁₁H₁₈FN₂O₂S | 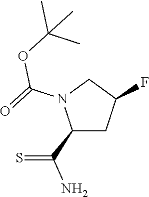 [SMILES: CC(C)(C)OC(=O)N1C[C@@H](F)C[C@H]1C(N)=S] | tert-butyl (2S,4S)-2- carbamothioyl-4- fluoropyrrolidine-1-carboxylate |
| 24 | C₁₂H₁₈F₂N₂O₂S | 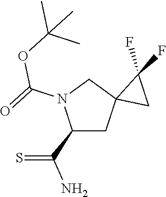 | tert-butyl (6S)-6-carbamothioyl- 1,1-difluoro-5- azaspiro[2.4]heptane-5- carboxylate |
| 25 | C₁₀H₁₈N₂O₂S | 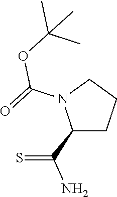 | tert-butyl (S)-2- carbamothioylpyrrolidine-1- carboxylate |
| 26 | C₁₁H₂₀N₂O₂S | 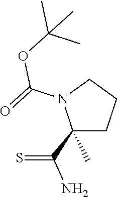 [SMILES: CC(C)(C)OC(=O)N1CCC[C@@]1(C)C(N)=S] | tert-butyl (S)-2-carbamothioyl-2- methylpyrrolidine-1-carboxylate |
| 27 | C₁₁H₂₀N₂O₃S | 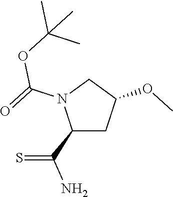 [SMILES: CO[C@@H]1C[C@@H](C(N)=S)N(C(=O)OC(C)(C)C)C1] | tert-butyl (2S,4R)-2- carbamothioyl-4- methoxypyrrolidine-1-carboxylate |
| 28 | C₁₁H₂₀N₂O₃S | 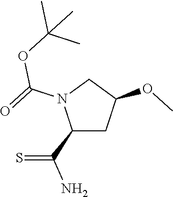 [SMILES: CO[C@H]1C[C@@H](C(N)=S)N(C(=O)OC(C)(C)C)C1] | tert-butyl (2S,4S)-2- carbamothioyl-4- methoxypyrrolidine-1-carboxylate |
| 29 | C₁₁H₂₀N₂O₂S | 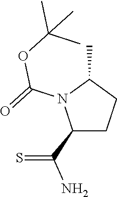 [SMILES: C[C@@H]1CC[C@@H](C(N)=S)N1C(=O)OC(C)(C)C] | tert-butyl (2S,5R)-2- carbamothioyl-5- methylpyrrolidine-1-carboxylate |
| 30 | C₁₁H₂₀N₂O₂S | 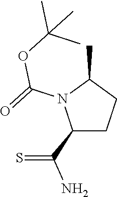 [SMILES: C[C@H]1CC[C@@H](C(N)=S)N1C(=O)OC(C)(C)C] | tert-butyl (2S,5S)-2- carbamothioyl-5- methylpyrrolidine-1-carboxylate |
| 31 | C₁₁H₂₀N₂O₂S | 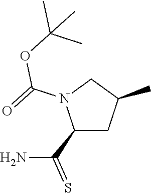 [SMILES: C[C@H]1C[C@@H](C(N)=S)N(C(=O)OC(C)(C)C)C1] | tert-butyl (2S,4S)-2- carbamothioyl-4- methylpyrrolidine-1-carboxylate |
| 32 | C₁₁H₁₇F₃N₂O₂S | 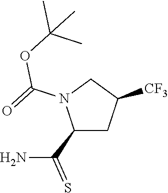 [SMILES: CC(C)(C)OC(=O)N1C[C@@H](C(F)(F)F)C[C@H]1C(N)=S] | tert-butyl (2S,4S)-2- carbamothioyl-4- (trifluoromethyl)pyrrolidine-1- carboxylate |
| 33 | C₂₆H₃₆N₂O₃SSi | 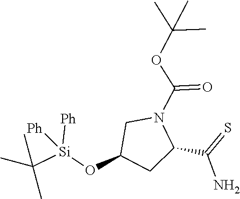 [SMILES: CC(C)(C)OC(=O)N1C[C@H](O[Si](c2ccccc2)(c2ccccc2)C(C)(C)C)C[C@H]1C(N)=S] | tert-butyl (2S,4R)-4-((tert- butyldiphenylsilyl)oxy)-2- carbamothioylpyrrolidine-1- carboxylate |
| 34 | C₂₆H₃₆N₂O₃SSi | 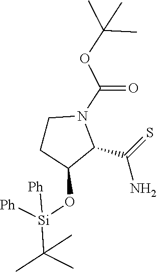 [SMILES: CC(C)(C)OC(=O)N1CC[C@H](O[Si](c2ccccc2)(c2ccccc2)C(C)(C)C)[C@H]1C(N)=S] | tert-butyl (2S,3S)-3-((tert- butyldiphenylsilyl)oxy)-2- carbamothioylpyrrolidine-1- carboxylate |
| 35 | C₁₁H₂₀N₂O₃S | 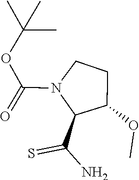 [SMILES: CO[C@H]1CCN(C(=O)OC(C)(C)C)[C@@H]1C(N)=S] | tert-butyl (2S,3S)-2- carbamothioyl-3- methoxypyrrolidine-1-carboxylate |
| 36 | C₁₂H₂₀N₂O₂S | 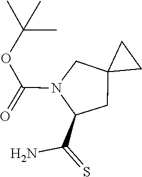 [SMILES: CC(C)(C)OC(=O)N1CC2(CC2)C[C@H]1C(N)=S] | tert-butyl (S)-6-carbamothioyl-5- azaspiro[2.4]heptane-5- carboxylate |
| 37 | C₂₆H₃₆N₂O₃SSi | 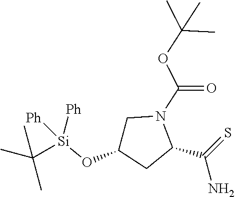 [SMILES: CC(C)(C)OC(=O)N1C[C@@H](O[Si](c2ccccc2)(c2ccccc2)C(C)(C)C)C[C@H]1C(N)=S] | tert-butyl (2S,4S)-4-((tert- butyldiphenylsilyl)oxy)-2- carbamothioylpyrrolidine-1- carboxylate |

TABLE 2b
Characterization of intermediate compounds (21) to (37)

| Compound | Yield | Characterization |
|---|---|---|
| 21 | 75% | [α]_{D} −52 (c 1.0, MeOH); ¹H NMR (500 MHz, DMSO) δ 9.78 (d, J = |
| | | 22.4 Hz, 1 H, NH), 9.39 (d, J = 26.1 Hz, 1 H, NH), 4.66-4.62 (m, 1 H, |
| | | H₅ₐ), 3.89-3.68 (m, 2 H, H_{5b} H₂), 2.97-2.79 (m, 1 H, H₃ₐ), 2.43-2.35 |
| | | (m, 1 H, H_{3b}), 1.41 (s, 3 H, CH_{3 9}), 1.36 (s, 6 H, 2 CH_{3 9}). ¹³C NMR (126 |
| | | MHz, DMSO) δ 203.1 (C₆), 152.4 (C₇), 113.8 (C₄), 80.5 (C₈), 63.8 (C₂), |
| | | 52.1 (C₅), 41.1 (C₃), 27.9 (C₉). HRMS ESI⁻ calculated 265.0911 for |
| | | C₁₀H₁₅F₂N₂O₂S, found 265.0822 |
| 22 | 83% | [α]_{D} −27 (c 1.0, MeOH); ¹H NMR (500 MHz, DMSO) δ 9.61 (d, J = |
| | | 41.2 Hz, 1 H, NH), 9.34 (d, J = 17.0 Hz, 1 H, NH), 5.28 (d, J_{H4-F} = 52.9 |
| | | Hz, 1 H, H₄), 4.55 (q, J = 8.1 Hz, 1 H, H₂), 3.77-3.49 (m, 2 H, H₅), 2.5- |
| | | 2.46 (m, 1 H, H₃ₐ) 2.16-1.99 (m, 1 H, H_{3b}), 1.39 (s, 3 H, CH_{3 9}), 1.35 (s, |
| | | 6 H, 2 CH_{3 9}). ¹³C NMR (126 MHz, DMSO) δ 208.3 (C₆), 170.4 (C₇), |
| | | 91.6 (q, C₄), 79.5 (C₈), 64.7 (C₂), 60.1 (C₅), 53.6 (C₃), 28.1 (C₉). HRMS |
| | | ESI⁺ calculated 249.1068 for C₁₀H₁₇F₁N₂O₂S, found 249.1065. |
| 23 | 49% | [α]_{D} −37 (c 1.0, MeOH); ¹H NMR (500 MHz, MeOD) δ 5.31-5.06 |
| | | (m, 1 H, H₄), 4.77-4.66 (m, 1 H, H₂), 3.90-3.75 (m, 1 H, H₅ₐ), 3.75- |
| | | 3.53 (m, 1 H, H_{5b}), 2.77-2.45 (m, 2 H, H₃), 1.48 (s, 3 H, CH_{3 9}), 1.44 |
| | | (s, 6 H, 2 CH_{3 9}). ¹³C NMR (126 MHz, DMSO) δ 207.0 (d, J = 12 Hz) |
| | | 206.5 (d, J = 12 Hz), 154.2, 153.8, 93.1 (d, J = 174 Hz), 92.0 (d, J = 174 |
| | | Hz), 79.7, 79.6, 66.6, 54.1 (d, J = 24 Hz), 53.7 (d, J = 24 Hz), 28.5. |
| | | HRMS ESI⁺ calculated 249.1068 for C₁₀H₁₈FN₂O₂S, found 249.1069. |
| 24 | 85% | [α]_{D} −35 (c 1.0, MeOH); ¹H NMR (500 MHz, MeOD) δ 4.81-4.61 (m, |
| | | 1 H, H₂), 3.81-3.57 (m, 2 H, H₅), 2.73-2.58 (m, 1 H, H₃ₐ), 2.20-2.06 |
| | | (m, 1 H, H_{3b}), 1.61-1.23 (m, 11 H, H₁₀ CH_{3 9}). ¹³C NMR (126 MHz, |
| | | DMSO) δ 208.4, 207.7, 153.5, 153.2, 114.3 (t, J = 288 Hz), 79.7, 66.5, |
| | | 66.4, 49.9, 49.2, 36.2, 35.7, 39.7, 29.7, 29.0 (t, J = 11 Hz), 20.1, 19.2 (t, |
| | | J = 11 Hz) HRMS ESI⁺ calculated 293.1130 for C₁₂H₁₇F₂N₂O₂S, found |
| | | 293.1128. |
| 25 | 81% | [α]_{D} −154 (c 0.18, EtOH); ¹H NMR (500 MHz, DMSO) δ 9.53 (s, 1H), |
| | | 9.10 (m, 1H), 4.44 (m, 1H), 3.45 (m, 1 H), 3.31 (m, 1H), 2.19 (m, 1H), |
| | | 1.80 (m, 3H), 1.39 (s, 3H), 1.33 (s, 6H). ¹³C NMR (126 MHz, DMSO) |
| | | δ 209.45, 208.69, 154.09, 153.62, 79.00, 66.85, 66.78, 47.44, 47.15, |
| | | 33.97, 33.18, 28.62, 28.49, 24.09, 23.48. HRMS ESI⁺ calculated |
| | | 253.0987 for C₁₀H₁₈N₂NaO₂S, found 253.0978. |
| 26 | 91% | [α]_{D} −60 (c 1.0, MeOH); ¹H NMR (500 MHz, MeOD) δ 3.62 (dt, J = |
| | | 10.3, 7.0 Hz, 1 H, H₅ₐ), 3.58-3.46 (m, 1 H, H_{5b}), 2.46-2.25 (m, 1 H, |
| | | H₃ₐ), 2.08-2.0 (m, 1 H, H_{3b}), 1.91-1.82 (m, 2 H, H₄), 1.74 (s, 3 H, |
| | | CH_{3 10}), 1.44 (s, 9 H, 2 CH_{3 9}). ¹³C NMR (126 MHz, MeOD) δ 212.0 (C₆), |
| | | 170.6 (C₇), 79.5 (C₈), 70.5 (C₂), 59.5 (C₅), 42.8 (C₃), 42.1 (C₄), 26.7(C₉), |
| | | 23.5 (C₉), 21.3 (C₁₀). HRMS ESI⁺ calculated 245.1318 for C₁₁H₂₁N₂O₂S, |
| | | found 245.1313. |
| 27 | 86% | [α]_{D} −22 (c 1.0, MeOH); ¹H NMR (500 MHz, MeOD) δ 4.68-4.53 (m, |
| | | 1 H, H₄), 4.07-3.92 (m, 1 H, H₂), 3.75-3.49 (m, 2 H, H₅), 3.34 (s, 3 H, |
| | | CH_{3 10}), 2.59-2.33 (m, 1 H, H₃ₐ), 2.05 (ddd, J_{H3b-H3a} = 13.4, J_{H3b-H2} = 8.4, |
| | | J_{H3b-H4} = 4.7 Hz, 1 H, H_{3b}), 1.46 (s, 3 H, CH_{3 9}), 1.43 (s, 6 H, 2 CH_{3 9}). |
| | | ¹³C NMR (126 MHz, MeOD) δ 209.3 (C₂), 154.3 (C₇), 77.7 (C₈), 65.0 |
| | | (C₄), 54.7 (C₂), 51.6 (C₁₀), 51.1 (C₅), 38.1 (C₃), 26.2 (C₉). HRMS ESI⁺ |
| | | calculated 261.1268 for C₁₁H₂₁N₂O₃S, found 261.1268. |
| 28 | 77% | [α]_{D} −22 (c 1.0, MeOH); ¹H NMR (500 MHz, MeOD) δ 4.66-4.53 (m, |
| | | 1 H, H₄), 3.99-3.88 (m, 1 H, H₂), 3.67-3.49 (m, 2 H, H₅), 3.27 (s, 3 H, |
| | | CH_{3 10}), 2.56-2.26 (m, 2 H, H₃), 1.47 (s, 3 H, CH_{3 9}), 1.43 (s, 6 H, 2 |
| | | CH_{3 9}). ¹³C NMR (126 MHz, DMSO) δ 208.2, 207.4, 154.3, 153.7, 79.4, |
| | | 78.6, 77.4, 66.1, 56.7, 56.5, 52.4, 51.6, 38.6, 37.7, 28.5, 28.4. HRMS |
| | | ESI⁺ calculated 261.1268 for C₁₁H₂₁N₂O₃S, found 261.1268. |
| 29 | 62% | [α]_{D} −58 (c 1.0, MeOH); ¹H NMR (500 MHz, DMSO) δ 9.51-9.46 (m, |
| | | 1H), 4.43 (d, J = 9 Hz, 1H), 4.08-4.01 (m, 1H), 2.33-2.25 (m, 1H), 2.22- |
| | | 2.15 (m, 1H), 1.70 (dd, J = 12 Hz, J = 7 Hz, 1H), 1.4 (m, 1H), 1.40 (s, 3H), |
| | | 1.33 (s, 6H), 1.12-1.08 (m, 3H). ¹³C NMR (126 MHz, DMSO) δ 209.6, |
| | | 208.7, 153.6, 153.1, 78.9, 66.9, 53.4, 31.1, 30.8, 30.33, 30.1, 28.6, 28.5, |
| | | 20.6, 19.8. HRMS ESI⁺ calculated 267.1143 for C₁₁H₂₀N₂O₂SNa, found |
| | | 267.1141. |
| 30 | 91% | [α]_{D} −14 (c 1.0, MeOH); ¹H NMR (500 MHz, DMSO) δ 9.55 (br s, 1H), |
| | | 8.96 (br s, 1H), 4.39 (t, J = 7 Hz), 3.85 (br s, 1H), 3.34 (m, 1H), 2.14- |
| | | 2.07 (m, 1H), 1.9 (m, 1H), 1.56-1.50 (m, 1H), 1.3 (m, 9H), 1.27 (d, J = 6 |
| | | Hz, 3H) ¹³C NMR (126 MHZ, DMSO) δ 209.5, 208.7, 154.6, 153.7, |
| | | 79.1, 68.2, 55.0, 54.7, 32.3, 31.7, 28.4, 21.6, 20.9. HRMS ESI⁺ |
| | | calculated 245.1318 for C₁₁H₂₁N₂O₂S, found 245.1318. |
| 31 | 94% | [α]_{D} −63 (c 1.0, MeOH); ¹H NMR (500 MHz, MeOD) δ 9.51-9.44 (m, |
| | | 1H), 9.18-9.13 (m, 1H) 4.39 (t, J = 9 Hz), 3.64 (t, J = 9 Hz), 2.97-2.89 |
| | | (m, 1H), 2.50-2.34 (m, 1H), 2.14-2.10 (m, 1H), 1.53-1.33 (m, 10H), |
| | | 0.97-0.95 (m, 3H). ¹³C NMR (126 MHz, DMSO) δ 209.6, 208.8, 153.8, |
| | | 153.4, 79.1, 67.5, 67.2, 54.7, 54.1, 42.5, 41.8, 32.9, 32.3, 28.6, 28.4, |
| | | 17.2, 17.1. HRMS ESI⁺ calculated 245.1318 for C₁₁H₂₁N₂O₂S, found |
| | | 245.1320. |
| 32 | 94% | [α]_{D} −58 (c 1.0, MeOH); ¹H NMR (500 MHz, MeOD) δ 9.67-9?59 (m, |
| | | 1H), 9.36-9.30 (m, 1H), 4.52 (t, J = 8 Hz, 1H), 3.79 (t, J = 9 Hz, 1H), |
| | | 3.48-3.29 (m, 2H), 2.56-2.53 (m, 1H), 1.94-1.85 (m, 1H), 1.40-1.35 (m, |
| | | 9H). ¹³C NMR (126 MHz, DMSO) δ 208.3, 207.5, 153.5, 153.2, 126.9 |
| | | (q, J = 277 Hz), 79.9, 65.9, 65.7, 46.7, 46.4, 33.4, 32.7, 28.5, 28.4. |
| | | HRMS ESI⁻ calculated 297.0885 for C₁₁H₁₆F₃N₂O₂S, found 297.0889. |
| 33 | 90% | [α]_{D} −14 (c 1.0, MeOH); ¹H NMR (500 MHz, MeOD) δ 7.71-7.57 |
| | | (m, 4 H, Hₐᵣₒ), 7.52-7.35 (m, 6 H, Hₐᵣₒ), 4.80 (t, J_{H4-H5} = J_{H4-H3} = 7.9 |
| | | Hz, 1 H, H₄), 4.45 (s, 1 H, H₂), 3.56 (dd, J = 26.7, 11.4 Hz, 1 H, H₅ₐ), |
| | | 3.40 (dd, J = 11.4, 3.6 Hz, 1 H, H_{5b}), 2.47-2.38 (m, 1 H, H₃ₐ), 1.93 |
| | | (ddd, J = 12.8, 8.6, 4.1 Hz, 1 H, H_{3b}), 1.45 (s, 6 H, 2 CH_{3 9}), 1.44 (s, 3 |
| | | H, CH_{3 9}), 1.06 (s, 9 H, 3 CH_{3 11}). ¹³C NMR (126 MHz, MeOD) 209.1 |
| | | (C₆), 154.5 (C₇), 134.8 (Cₐᵣₒ), 132.6 (Cₐᵣₒ), 129.1 (Cₐᵣₒ), 126.9 (Cₐᵣₒ), 79.8 |
| | | (C₈), 70.5 (C₂), 65.6 (C₄), 54.5 (C₅), 47.2 (C₃), 42.4 (C₁₀), 26.6 (C₉), 25.4 |
| | | (C₁₁). HRMS ESI⁺ calculated 485.2289 for C₂₆H₃₇N₂O₃SSi, found |
| | | 485.2284. |
| 34 | 86% | [α]_{D} +16 (c 1.0, MeOH); ¹H NMR (500 MHz, MeOD) δ 9.71 (br s, |
| | | 1H), 9.16-9.10 (m, 1H), 7.65 (d, J = 7 Hz, 2H), 7.61 (d, J = 7 Hz), 7.49- |
| | | 7.41 (m, 6H), 6.51-6.38 (m, 2H), 4.54-4.52 (m, 1H), 3.68-3.67 (m, 2H), |
| | | 3.50-3.48 (m, 1H), 1.72-1.59 (m, 2H), 1.44-1.36 (m, 9H), 1.03 (bs, 9H). |
| | | ¹³C NMR (126 MHz, DMSO) δ 204.6, 203.8, 154.9, 154.2, 135.9, |
| | | 135.8, 135.7, 135.6, 133.7, 133.6, 133.2, 133.1, 130.5, 128.4, 128.3, |
| | | 79.5, 79.4, 79.3, 78.6, 76.6, 76.4, 69.8, 45.4, 45.0, 32.6, 31.9, 28.6, |
| | | 28.5, 28.1, 27.1, 19.4. HRMS ESI⁺ calculated 485.2249 for |
| | | C₂₆H₃₇N₂O₃SSi, found 285.2294. |
| 35 | 80% | [α]_{D} −15 (c 1.0, MeOH); ¹H NMR (500 MHz, MeOD) δ 9.72 (br s, 1H) |
| | | 9.29-9.24 (m, 1H) 4.41 (br d, J = 16 Hz, 1H) 3.80 (br d, J = 4 Hz, 1H), |
| | | 3.56 (t, J = 9 Hz, 1H), 3.28-3.24 (m, 1H) 2.02-1.97 (m, 1H), 1.89-1.83 |
| | | (m, 1H), 1.40, 1.35 (m, 9H). ¹³C NMR (126 MHz, DMSO) δ 205.6, |
| | | 204.9, 154.3, 153.7, 86.6, 85.8, 79.4, 79.3, 56.5, 56.4, 45.7, 45.4, 29.7, |
| | | 29.0, 28.6, 28.5. HRMS ESI⁻ calculated 259.1126 for C₁₁H₁₉N₂O₃S, |
| | | found 259.1123. |
| 36 | 82% | [α]_{D} −21 (c 1.0, MeOH); ¹H NMR (500 MHz, MeOD) δ 9.60 (br s, 1H), |
| | | 9.15-9.09 (m, 1H), 4.55-4.52 (m, 1H), 3.39-3.34 (m, 3H), 2.26 (ddd, J = |
| | | 47 Hz, J = 12 Hz, J = 6 Hz, 1H), 1.79 (ddd, J = 39 Hz, J = 12 Hz, J = |
| | | 3 Hz), 1.40-1.35 (m, 9H), 0.62-0.44 (m, 4H). ¹³C NMR (126 MHz, |
| | | DMSO) δ 208.8, 208.1, 154.1, 153.6, 79.2, 67.8, 54.8, 54.4, 42.1, 41.4, |
| | | 28.6, 28.5, 20.8, 20.4, 12.5, 11.0, 9.7, 8.2 HRMS ESI⁺ calculated |
| | | 279.1143 for C₁₂H₂₀N₂O₂SNa, found 279.1138. |
| 37 | 76% | [α]_{D} −8 (c 1.0, MeOH); ¹H NMR (500 MHz, MeOD) δ 7.66 (dd, J = 7.9, |
| | | 1.4 Hz, 4 H, Hₐᵣₒ), 7.54-7.37 (m, 6 H, Hₐᵣₒ), 4.42-4.30 (m, 1 H, H₄), |
| | | 4.22-4.07 (m, 1 H, H₂), 3.55-3.36 (m, 2 H, H₅), 2.34-2.03 (m, 2 H, |
| | | H₃), 1.42 (s, 9 H, 3 CH_{3 11}), 1.05 (s, 9 H, 3 CH_{3 9}). ¹³C NMR (126 MHz, |
| | | MeOD) δ 208.4 (C₆), 176.6 (C₇), 154.8 (Cₐᵣₒ), 135.5 (Cₐᵣₒ), 133.4 (Cₐᵣₒ), |
| | | 129.5 (Cₐᵣₒ), 127.7 (Cₐᵣₒ), 80.1 (C₈), 71.7(C₂), 70.5 (C₄), 59.0 (C₅), 53.8 (C₃), |
| | | 38.7 (C₁₀), 27.1 (C₉), 25.6 (C₉), 18.4 (C₁₁). HRMS ESI⁺ calculated |
| | | 485.2289 for C₂₆H₃₇N₂O₃SSi, found 485.2286. |

All the intermediates and compounds described hereinafter can be synthetized according to schemes 1 and 2.

The examples that follow describe the preparation of certain intermediate compounds and of compounds of formula (I). The examples are not limiting but serve merely to illustrate the present invention.

In the following examples, when the source of the starting products is not specified, it should be understood that said products are known products.

The following abbreviations and empirical formulae are used:
NEt₃ triethylamine
DMAP 4-Dimethylaminopyridine
NaHCO₃Sodium bicarbonate
DCM Dichloromethane
Na₂SO₄ Sodium sulfate
EtOAc Ethyl acetate
r.t. Room temperature
MgSO₄ Magnesium sulfate
THF Tetrahydrofuran
N₂ Nitrogen
DMSO Dimethyl sulfoxide
KHSO₄ Potassium bisulfate
TBAF Tetra-n-butylammonium fluoride
tBuMgCl Tert-butyl magnesium chloride
NH₄OH Ammonium hydroxide
DMF Dimethylformamide.

### EXAMPLES

### Example 1: Synthesis of Intermediate Compounds 21 to 26, 29 to 32 and 36

### Intermediate 26: Tert-butyl(S)-2-carbamothioyl-2-methylpyrrolidine-1-carboxylate

Intermediate 26 can be obtained from(S)-1-(tert-butoxycarbonyl)-2-methylpyrrolidine-2-carboxylic acid by a process as described below.

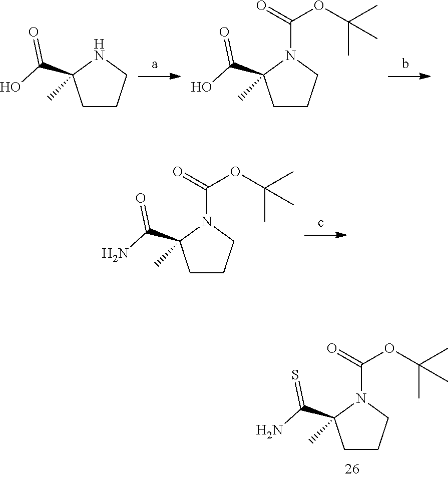

### Step a: (S)-1-(tert-butoxycarbonyl)-2-methylpyrrolidine-2-carboxylic acid

To a solution of commercially available(S)-2-methylpyrrolidine-2-carboxylic acid (400 mg, 3.10 mmol, 1 equiv) in DCM (15 mL) was successively added, at 0° C., triethylamine (648 μL, 4.65 mmol, 1.5 eq), di-tert-butyl dicarbonate (853 μL, 3.72 mmol, 1.2 equiv.), and DMAP (57 mg, 464 μmol, 0.15 equiv.). The reaction mixture was stirred at 0° C. for 30 min and at room temperature overnight. The reaction was quenched with NaHCO₃ saturated aqueous solution (15 mL) and extracted with DCM (3×15 mL). Organic phase was dried over Na₂SO₄ anhydrous, filtered, and concentrated in vacuo. Flash chromatography of the residue (Cyclo/EtOAc 5/5); afforded (2S)-1-(tert-butoxycarbonyl)-2-methylpyrrolidine-2-carboxylic acid as a colorless oil (461 mg, 65% yield):

R_{f} 0.60 (Cyclo/EtOAc 1/4); [α]_{D}-8 (c 1.0, MeOH); ¹H NMR (500 MHz, MeOD) δ 3.61-3.39 (m, 2H, H₅), 2.38-2.10 (m, 1H, H₃ₐ), 2.04-1.79 (m, 3H, H_{3b}, H₄), 1.51 (d, J=13.3 Hz, 3H, H₁₀), 1.44 (s, 3H, CH_{3 9}), 1.42 (s, 6H, 2 CH_{3 9}). ¹³C NMR (126 MHz, MeOD) δ 178.2 (C₆), 155.7 (C₇), 81.6 (C₈), 65.9 (C₂), 54.7 (C₅), 41.3 (C₃), 28.5 (C₉), 23.2 (C₄), 22.5 (C₁₀). HRMS ESI⁺ calculated 252.1206 for C₁₁H₁₉O₄NNa. found 252.1204.

### Step b: tert-butyl-(2S)-2-carbamoyl-2-methylpyrrolidine-1-carboxylate

To a solution of (2S)-1-(tert-butoxycarbonyl) 2-methylpyrrolidine-2-carboxylic acid (350 mg, 1.53 mmol, 1 equiv) in dry THF (5 mL) was successively added at −10° C. under Argon, chloroformate isobutyl (458 μL, 3.51 mmol, 2.3 equiv.) and triethylamine (212 μL, 1.53 mmol, 1 equiv.). The reaction mixture was stirred at 0° C. for 30 min. 0.75 mL of ammoniac solution (28% in water) was added at −10° C. and the reaction mixture was stirred at r.t. overnight. Solvent were removed in vacuo. The residue was dissolved into AcOEt (15 mL) filtered then dried over MgSO₄ anhydrous and concentrated in vacuo. Flash chromatography of the residue (Cyclo/AcOEt 1/1; afforded tert-butyl-(2S)-2-carbamoyl-2-methylpyrrolidine-1-carboxylate as a white powder (240 mg, 60% yield):

R_{f} 0.15 (Cyclo/AcOEt 1/1); [α]_{D}-23 (c 1.0, MeOH); ¹H NMR (500 MHz, MeOD) δ 3.59 (dt, J=10.3, 7.0 Hz, 1H), 3.47 (dt, J_{H5a-H5b}=10.4, J_{H5a-H4}=6.4 Hz, 1H, H₅ₐ), 2.20 (dt, J_{H5b-H5a}=13.0, J_{H5b-H4}=7.4 Hz, 1H, H₅ₐ), 2.03-1.93 (m, 1H, H₃ₐ), 1.96-1.85 (m, 3H, H_{3b}, H₄), 1.53 (d, J=16.3 Hz, 3H, CH_{3 10}), 1.45 (d, J=6.5 Hz, 9H, 3 CH₃). ¹³C NMR (126 MHz, MeOD) δ 180.6 (C₆), 155.4 (C₇), 81.7 (C₈), 67.1 (C₂), 42.1 (C₅), 41.6 (C₃), 28.7 (C₉), 23.4 (C₄), 22.9 (C₁₀). HRMS ESI⁺ calculated 251.1366 for C₁₁H₂₀O₃N₂Na, found 251.1359.

### Step c: tert-butyl(S)-2-carbamothioyl-2-methylpyrrolidine-1-carboxylate

To a solution of tert-butyl-(2S)-2-carbamoyl-2-methylpyrrolidine-1-carboxylate (210 mg, 920 μmol, 1 equiv) in dry THF (2 mL) was added Lawesson's reagent (186 mg, 460 μmol, 0.5 equiv.) under Argon. The reaction mixture was stirred at r.t. for 3.5 hours. Solvent were removed in vacuo. Flash chromatography of the residue (Cyclo/AcOEt 1/1); afforded the intermediate 26 as a white powder (204 mg, 91% yield). Characterization is detailed in Table 2b.

Similar processes were performed starting from commercially available starting compounds to obtain the intermediates 21 to 25, 29 to 32 and 36. Characterization of the obtained intermediate compounds are given in Table 2b.

### Example 2: Synthesis of Intermediate Compounds 27, 28 and 35

Intermediate 28: Tert-butyl(2S,4S)-2-carbamothioyl-2-methoxypyrrolidine-1-carboxylate Intermediate 28 can be obtained from (4S)-4-hydroxypyrrolidine-2-carboxylic acid by a process as described below.

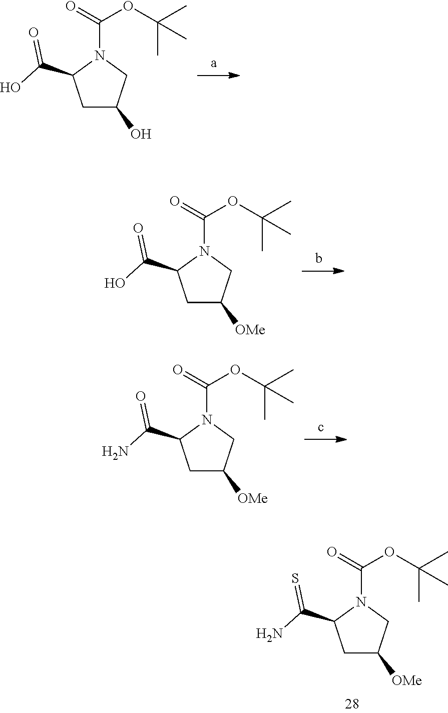

### Step a: (2S,4S)-1-(tert-butoxycarbonyl)-4-methoxypyrrolidine-2-carboxylic acid

NaH (55% in mineral oil, 216 mg, 5.4 mmol, 2.5 eq) was placed in a flame-dried two-necked round bottom flask, equipped with stirring bar, N₂ inlet, and rubber septum; the oil was eliminated by rinsing the hydride with anhydrous pentane (3×10 mL) under N₂; the resulting white solid was gently fluxed with a N₂ stream until complete dryness. After cooling the flask at −20° C. under N₂, a solution of N-Boc-cis-4-hydroxy-L-proline (500 mg, 2.0 mmol, 1 eq) in a mixture of anhydrous THF (2 mL) and DMSO (0.2 mL) was slowly added by syringe. The temperature was then raised to reach 0° C. and the heterogeneous mixture was stirred for 30 min. The temperature was reduced again to −20° C. and a solution of methyl iodide (333 μL, 5.4 mmol, 2.5 eq) in THF (1.2 mL) was added by syringe. Finally, the temperature was set at 25° C. and the reaction mixture was stirred until the starting material was completely consumed (TLC analysis). The reaction was then quenched with water (2 mL) and the pH was adjusted to 4 with 10% aqueous solution of KHSO₄ (ca. 2 mL); the crude was extracted with EtOAc (5×6 mL); the organic layer was washed with brine (3×4 mL), dried over MgSO₄, and the solvent were removed in vacuo. Flash chromatography of the residue (DCM/MeOH 95/5); afforded (2S,4S)-1-(tert-butoxycarbonyl)-4-methoxypyrrolidine-2-carboxylic acid as a white powder (400 mg, 75% yield):

R_{f} 0.36 (DCM/MeOH 95/5); [α]_{D}-63 (c 1.0, MeOH); ¹H NMR (500 MHz, MeOD) δ 4.34-4.26 (m, 1H, H₄), 4.0-3.95 (m, 1H, H₂), 3.65-3.55 (m, 1H, H₅ₐ), 3.47-3.39 (m, 1H, H_{5b}), 3.28-3.23 (m, 3H, H₁₀), 2.42-2.38 (m, 1H, H₃ₐ), 2.34-2.19 (m, 1H, H_{3b}), 1.48 (s, 3H, CH_{3 9}), 1.43 (s, 6H, 2 CH_{3 9}). ¹³C NMR (126 MHz, MeOD) δ 175.8 (C₆), 156.0 (C₇), 81.2 (C₄), 80.0 (C₈), 58.6 (C₂), 52.8 (C₁₀), 52.0 (C₅), 35.9 (C₃), 28.5 (C₉). HRMS ESI⁺ calculated 268.1155 for C₁₁H₁₉O₅NNa. found 268.1152.

### Step b: tert-butyl-(2S,4S)-2-carbamoyl-4-methoxypyrrolidine-1-carboxylate

To a solution of (2S,4S)-1-(tert-butoxycarbonyl)-4-methoxypyrrolidine-2-carboxylic acid (400 mg, 1.63 mmol, 1 equiv) in dry THF (2 mL) was successively added at −10° C. under Argon, chloroformate isobutyl (488 μL, 3.75 mmol, 2.3 equiv.) and triethylamine (227 μL, 1.63 mmol, 1 equiv.). The reaction mixture was stirred at 0° C. for 30 min. 0.75 mL of ammonia (28% in water) was added at −10° C. and the reaction mixture was stirred at r.t. overnight. Solvent were removed in vacuo. The residue was dissolved into AcOEt (15 mL), filtered then dried over MgSO₄ anhydrous and concentrated in vacuo. Flash chromatography of the residue (Cyclo/AcOEt 1/1; afforded tert-butyl-(2S,4S)-2-carbamoyl-4-methoxypyrrolidine-1-carboxylate as a white powder (292 mg, 73% yield):

R_{f} 0.25 (Cyclo/AcOEt 1/4); [α]_{D}-22 (c 1.0, MeOH); ¹H NMR (500 MHz, MeOD) δ 4.26-4.13 (m, 1H, H₂), 4.0-3.97 (m, 1H, H₅ₐ), 3.59 (t, J=11.5, 1H, H₄), 3.52-3.49 (m, 1H, H_{5b}), 3.29 (s, 3H, CH_{3 10}), 2.47-2.09 (m, 2H, H₃), 1.48 (s, 3H, CH₃), 1.46 (d, J=17.6 Hz, 6H, 2 CH₃). ¹³C NMR (126 MHz, MeOD) δ 178.4 (C₆), 156.1 (C₇), 80.4 (C₄), 79.5 (C₈), 60.8 (C₂), 53.7 (C₁₀), 52.8 (C₅), 36.7 (C₃), 28.6 (C₉), 28.6 (C₉). HRMS ESI⁺ calculated 267.1315 for C₁₁H₂₀O₄N₂Na. found 267.1313.

### Step c: tert-butyl(2S,4S)-2-carbamothioyl-4-methoxypyrrolidine-1-carboxylate

To a solution of tert-butyl-(2S,4S)-2-carbamoyl-4-methoxypyrrolidine-1-carboxylate (280 mg, 1.15 mmol, 1 equiv) in dry THF (2 mL) was added Lawesson's reagent (231 mg, 573 μmol, 0.5 equiv.) under Argon. The reaction mixture was stirred at r.t. for 3.5 hours. Solvent was removed in vacuo. Flash chromatography of the residue (Cyclo/AcOEt 1/1); afforded the intermediate compound 28 as a white powder (219 mg, 77% yield). Characterization is detailed in Table 2b.

Similar processes were performed starting from commercially available starting compounds to obtain the intermediates 27 and 35. Characterization of the obtained intermediate compounds are given in Table 2b.

### Example 3: Synthesis of Intermediate Compounds 33, 34 and 37

### Intermediate 33: tert-butyl(2S,4R)-4-((tert-butyldiphenylsilyl)oxy)-2-carbamothioylpyrrolidine-1-carboxylate

Intermediate 33 can be obtained from (2S,4R)-1-(tert-butoxycarbonyl)-4-hydroxypyrrolidine-2-carboxylic acid by a process as described below.

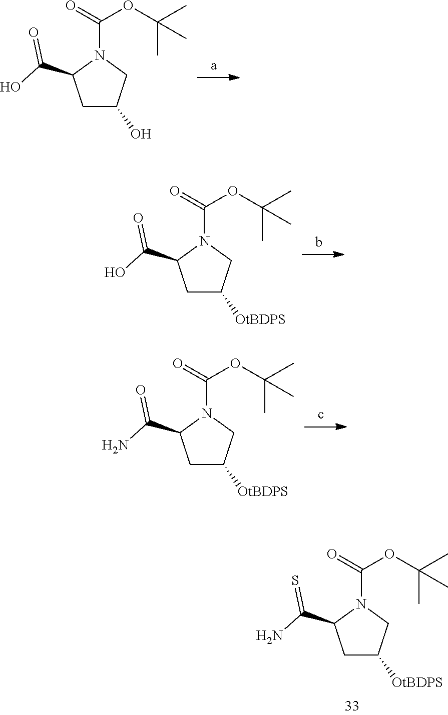

### Step (a): N-Boc-trans-4-(OtBDPS)-L-proline

To a solution of N-Boc-trans-4-hydroxy-L-proline (585 mg, 2.53 mmol, 1 equiv) in dry DMF (7 mL) was successively added at 0° C. under Argon, imidazole (775 mg, 11.4 mmol, 4.5 equiv.) and tert-Butylchlorodiphenylsilane (986 μL, 3.8 mmol, 1.5 equiv.). The reaction mixture was stirred at r.t. for 5 days. 30 mL of water was added at 0° C. and the reaction mixture was acidified to pH2 with HCl 1M, extracted with EtOAc (5×15 mL), dried over MgSO₄ anhydrous, filtered and concentrated in vacuo. Flash chromatography of the residue (Cyclo/EtOAc 9/1); afforded N-Boc-trans-4-(OtBDPS)-L-proline as a white powder (480 mg, 69% yield):

R_{f} 0.10 (Cyclo/EtOAc 9/1); [α]_{D}-14 (c 1.0, MeOH); ¹H NMR (500 MHz, MeOD) δ 7.71-7.59 (m, 4H, Hₐᵣₒ), 7.58-7.35 (m, 6H, Hₐᵣₒ), 4.51-4.43 (m, 1H, H₂), 4.38 (dt, J_{H4-H5}=23.1, J_{H4-H3}=8.0 Hz, 1H), 3.48-3.39 (m, 1H, H₅ₐ), 3.31 (ddd, J_{H5b-H5a}=39.5, 11.3, 4.0 Hz, 1H, H_{5b}), 2.40-2.24 (m, 1H, H₃ₐ), 1.96-1.86 (m, 1H, H₃ᵥ), 1.44 (s, 3H, CH_{3 9}), 1.43 (s, 6H, 2 CH_{3 9}), 1.06 (s, 9H, 3 CH_{3 11}). ¹³C NMR (126 MHz, MeOD) δ 176.4 (C₆), 156.7 (C₇), 137.5 (Cₐᵣₒ), 134.3 (Cₐᵣₒ), 131.5 (Cₐᵣₒ), 129.2 (Cₐᵣₒ), 82.4 (C₈), 73.0 (C₄), 72.5 (C₂), 59.6 (C₃), 55.6 (C₅), 41.1 (C₁₀), 28.5 (C₉), 27.3 (C₁₁). HRMS ESI⁺ calculated 470.2357 for C₂₆H₃₆NO₅NSi. found 470.2354.

### Step (b): tert-butyl(2S,4R)-4-((tert-butyldiphenylsilyl)oxy)-2-carbamoylpyrrolidine-1-carboxylate

To a solution of N-Boc-trans-4-(OtBDPS)-L-proline (700 mg, 1.5 mmol, 1 equiv) in dry THF (4 mL) was successively added at −10° C. under Argon, chloroformate isobutyl (446 μL, 3.4 mmol, 2.3 equiv.) and triethylamine (208 μL, 1.5 mmol, 1 equiv.). The reaction mixture was stirred at 0° C. for 30 min. 0.75 mL of ammonia (28% in water) was added at −10° C. and the reaction mixture was stirred at r.t. overnight. Solvent were removed in vacuo. The residue was dissolved into AcOEt (15 mL) filtered then dried over MgSO₄ anhydrous and concentrated in vacuo. Flash chromatography of the residue (Cyclo/AcOEt 1/1; afforded Tert-butyl(2S,4R)-4-((tert-butyldiphenylsilyl)oxy)-2-carbamoylpyrrolidine-1-carboxylate as a white powder (662 mg, 94% yield):

R_{f} 0.35 (Cyclo/AcOEt 1/1); [α]_{D}-9 (c 1.0, MeOH); ¹H NMR (500 MHz, MeOD) δ 1H NMR (500 MHz, MeOD) δ 7.67-7.61 (m, 4H, Hₐᵣₒ), 7.48-7.39 (m, 6H, Hₐᵣₒ), 4.47-4.44 (m, 1H, H₂), 4.42-4.34 (m, 1H, H₄), 3.53-3.43 (m, 1H, H₅ₐ), 3.36 (dd, J_{H5b-H5a}=11.4, J_{H5b-H4}=3.8 Hz, 1H, H_{5b}), 2.37-2.10 (m, 1H, H₃ₐ), 1.89 (ddd, J_{H3b-H3a}=12.9, J_{H3b-H4}=8.8, J_{H3b-H2}=4.2 Hz, 1H, H_{3b}), 1.46 (s, 6H, 2 CH_{3 9}), 1.44 (s, 3H, CH_{3 9}), 1.06 (s, 9H, CH_{3 11}). ¹³C NMR (126 MHz, MeOD) δ 178.4 (C₆), 156.3 (C₇), 136.8 (Cₐᵣₒ), 134.6 (Cₐᵣₒ), 131.2 (Cₐᵣₒ), 128.6 (Cₐᵣₒ), 81.5 (C₄), 73.1 (C₈), 72.6 (C₁₀), 61.1 (C₂), 56.2 (C₅), 41.0 (C₃), 28.8 (C₉), 27.5 (C₉), 19.9 (C₁₁), HRMS ESI⁺ calculated 469.2517 for C₂₆H₃₇O₄N₂Si, found. 469.2515.

### Step (c): Tert-butyl(2S,4R)-4-((tert-butyldiphenylsilyl)oxy)-2-carbamothioylpyrrolidine-1-carboxylate

To a solution of Tert-butyl(2S,4R)-4-((tert-butyldiphenylsilyl)oxy)-2-carbamoylpyrrolidine-1-carboxylate (650 mg, 917 μmol, 1 equiv) in dry THF (2 mL) was added Lawesson's reagent (280 mg, 693 μmol, 0.5 equiv.) under Argon. The reaction mixture was stirred at r.t. for 3.5 hours. Solvent were removed in vacuo. Flash chromatography of the residue (gradient DCM/MeOH 100/0 to 98/2) afforded the intermediate compound 33 as a colorless oil (609 mg, 90% yield). Characterization is detailed in Table 2b.

Similar processes were performed starting from commercially available starting compounds to obtain the intermediates 34 and 37. Characterization of the obtained intermediate compounds are given in Table 2b.

### Example 4: Synthesis of Compounds 1 to 12, 15 and 16

Compound 5: (S)-2-carbamothioyl-N-(4-methyl-5-(2-(1,1,1-trifluoro-2-methylpropan-2-yl)pyridin-4-yl)thiazol-2-yl)pyrrolidine-1-carboxamide Compound 5 can be obtained from 4-methyl-5-[2-(2,2,2-trifluoro-1,1-dimethylethyl)-4-pyridinyl]-2-thiazolamine and intermediate compound 25, by a process as described below.

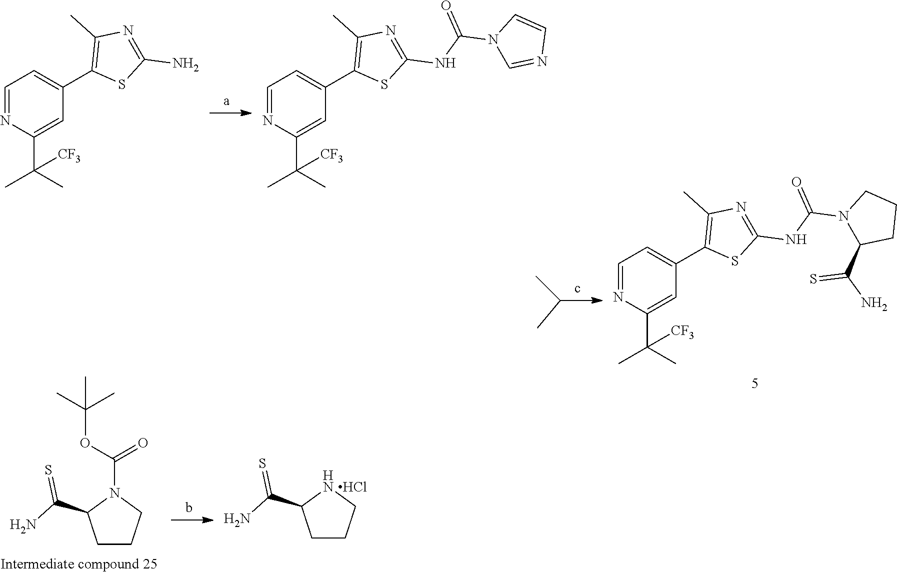 [SMILES: Cc1nc(N)sc1-c1ccnc(C(C)(C)C(F)(F)F)c1 ; Cc1nc(NC(=O)n2ccnc2)sc1-c1ccnc(C(C)(C)C(F)(F)F)c1 ; Cc1nc(NC(=O)N2CCC[C@H]2C(N)=S)sc1-c1ccnc(C(C)(C)C(F)(F)F)c1 ; CC(C)(C)OC(=O)N1CCC[C@H]1C(N)=S ; NC(=S)[C@@H]1CCCN1 ; Cl]

### Step (a): N-{4-methyl-5-[2-(1,1,1-trifluoro-2-methylpropan-2-yl)pyridine-4-yl]-1,3-thiazol-2-yl}-1H-imidazole-1-carboxamide

4-Methyl-5-[2-(2,2,2-trifluoro-1,1-dimethylethyl)-4-pyridinyl]-2-thiazolamine is commercially available from AURUM pharmatech.

4-Methyl-5-[2-(2,2,2-trifluoro-1,1-dimethylethyl)-4-pyridinyl]-2-thiazolamine (2 g, 6.637 mmol) was dissolved in methylene chloride (60 mL) and 1,1′-carbonyldimidazole (1.867 g, 11.512 mmol) was added. The reaction mixture was heated under reflux for 14 h, cooled to room temperature and filtrated. The amorphous solid obtained (2.62 g, 95% yield) was used in the next step without further purification.

### Step (b): (S)-pyrrolidine-2-carbothioamide hydrochloride

Intermediate compound 25 was dissolved at 0° C. in a dioxane solution of HCl (4N, 90 mL) and stirred at 0° C. for one hour. The reaction mixture was then let to warm up to room temperature and solvent was evaporated under vacuum. The white solid was triturated in diethylether and filtrated to deliver (2S)-pyrrolidine-2-carbothioamide hydrochloride (5.43 g, quantitative).

### Step (c): (S)-2-carbamothioyl-N-(4-methyl-5-(2-(1,1,1-trifluoro-2-methylpropan-2-yl)pyridin-4-yl)thiazol-2-yl)pyrrolidine-1-carboxamide

N-{4-methyl-5-[2-(1,1,1-trifluoro-2-methylpropan-2-yl)pyridine-4-yl]-1,3-thiazol-2-yl}-1H-imidazole-1-carboxamide (365 mg) and(S)-pyrrolidine-2-carbothioamide hydrochloride (156 mg) were added to DMF (7 mL) and Et₃N (385 μL) was added dropwise to the reaction mixture, leading to an homogeneous solution. The reaction mixture was stirred for 14 h at room temperature and quenched by water (100 mL). The reaction mixture was extracted by dichloromethane (3×30 mL), the organic phase was washed with brine and dried over Na₂SO₄. Solvent evaporation under vacuum led to a yellow oil that was purified by reverse HPLC (gradient MeCN/H₂O 45/55 to 95/5 in 25 min. 90 ml·min⁻¹). 300 mg of compound 5 (73%) could be obtained. Characterization is detailed in Table 1b.

Similar processes were performed using intermediates compounds 21 to 24, 26 to 32, 35 and 36 to obtain compounds 1 to 4, 6 to 12, 15 and 16. Characterization of the obtained compounds are given in Table 1b.

### Example 5: Synthesis of Compounds 13, 14 and 17

### Compound 17: (2S,4S)-2-carbamothioyl-4-hydroxy-N-(4-methyl-5-(2-(1,1,1-trifluoro-2-methylpropan-2-yl)pyridin-4-yl)thiazol-2-yl)pyrrolidine-1-carboxamide

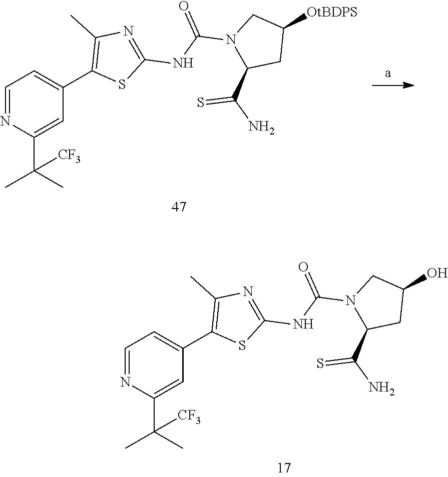

Compound 47 is obtained as detailed in example 4 above, from intermediate compound 37 and 4-Methyl-5-[2-(2,2,2-trifluoro-1,1-dimethylethyl)-4-pyridinyl]-2-thiazolamine.

Compound 47 (210 mg) was dissolved in THF (5 mL) and TBAF (1M in THE, 2.5 mL) was added. The solution was stirred at room temperature for 14 h, and water (45 mL) was added. The reaction mixture was extracted by dichloromethane (3×25 mL) and organic phases were washed with brine and dried over Na₂SO₄. The yellow oil was purified by preparative reverse HPLC (gradient MeCN/H₂O 45/55 to 95/5 in 25 min. 90 ml·min⁻¹) to yield 75 mg of compound 17 (75 mg). Characterization is detailed in Table 1b.

Similar processes were performed using intermediates compounds 33 and 34 to obtain compounds 13 and 14. Characterization of the obtained compounds are given in Table 1b.

### Example 6: Synthesis of Compounds (18), (19) and (20)

### 6.1. Synthesis of 5-(2-(tert-butyl)pyridin-4-yl)-4-methylthiazol-2-amine

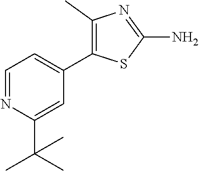 [SMILES: Cc1nc(N)sc1-c1ccnc(C(C)(C)C)c1]

### Step (a): Synthesis of 2-tert-butyl-4-methylpyridine

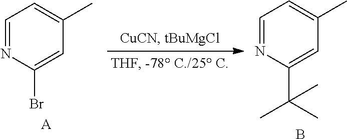

A dried, Argon flushed 250 ml schlenk was placed with CuCN (dried at 140° C. during 3 h, 3.22 g, 36 mmol, 4 equiv) and anhydrous THF (90 ml). The mixture was cooled at −78° C., and 72 ml of tBuMgCl (1 M in THF, 8 equiv) was added dropwise. Then, the mixture was stirred at −78° C. for 20 min before adding 2-bromo-4-methylpyridine (1.55 g, 9 mmol, 1.0 equiv). The reaction mixture was stirred at −78° C. for 3 h and then warmed to room temperature for 12 h. The resulting mixture was quenched with saturated aqueous NH₄OH (40 ml), and the pH was adjusted to 10 by 1 M aqueous NaOH (55 ml). After filtration on Celite, the resulting solution was extracted with Et₂O (75 ml×3). The combined organic layers were dried over MgSO₄, and solvent was removed under reduced pressure. The residue was purified by flash column chromatography on silica gel (Cyclohexane/EtOAc=9/1 v/v) to give the corresponding product as yellow oil (0.66 g, 49% yield):

R_{f} 0.35 (Cyclo/AcOEt 9/1); ¹H NMR (500 MHz, DMSO) δ 8.35 (d, J=4.9 Hz, 1H), 7.29-7.16 (m, 1H), 7.07-6.91 (m, 1H), 2.29 (s, 3H), 1.28 (s, 9H). ¹³C NMR (126 MHz, DMSO) δ 168.7, 148.5, 147.2, 122.2, 120.1, 37.5, 30.5, 21.2.

### Step (b): Synthesis of 1-(2-tert-butylpyridin-4-yl) propan-2-one

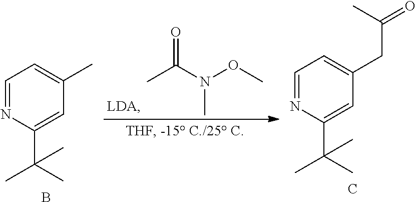

A dried, Argon flushed 25 ml three-neck flask was placed with a freshly prepared LDA solution (6.63 mmol in 4 ml of anhydrous THF, 1.5 equiv). The mixture was cooled at −15° C. and a solution of 2-tert-butyl-4-methylpyridine (0.66 g, 4.42 mmol, 1 equiv) in 4 ml of anhydrous THF was added dropwise. Then, the mixture was stirred at −15° C. for 30 minutes before adding dropwise N-methoxy-N-methylacetamide (0.524 g, 5.09 mmol, 1.15 equiv) in 3 ml of anhydrous THF. The reaction was stirred at −15° C. for 1 h30 before being transferred onto a mixture of 5 ml of aqueous sulfuric acid 1.5M and 20 ml of toluene at 0° C. The biphasic mixture was vigorously stirred at 0° C. for 25 minutes. Before phase separation the pH was adjusted to 7 using saturated NaHCO₃ aqueous solution. The aqueous phase was extracted with DCM (50 ml×2) and the combined organic phase dried over MgSO₄. The solvent was removed under reduced pressure. The residue was purified by column chromatography on silica gel (Cyclohexane/EtOAc 75/25 v/v) to give the corresponding product as a yellow oil (0.5 g, 59% yield):

R_{f} 0.22 (Cyclo/AcOEt 75/25); ¹H NMR (500 MHz, DMSO) δ 8.41 (dd, J=4.9, 0.5 Hz, 1H), 7.23 (d, J=0.5 Hz, 1H), 7.00 (dd, J=4.9, 1.5 Hz, 1H), 3.83 (s, 2H), 2.17 (s, 3H), 1.29 (s, 9H). ¹³C NMR (126 MHz, DMSO) δ 205.4, 168.7, 148.5, 144.3, 122.8, 120.7, 49.3, 37.6, 30.5, 30.3.

Step (c): Synthesis of 5-(2-(tert-butyl)pyridin-4-yl)-4-methylthiazol-2-amine

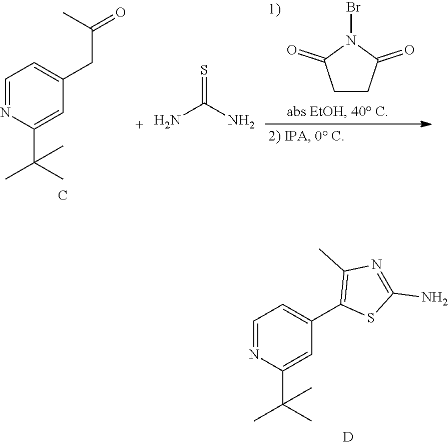

To a solution of 1-(2-tert-butylpyridin-4-yl) propan-2-one (0.5 g, 2.61 mmol, 1 equiv) in 7.5 ml of absolute ethanol was added thiourea (0.22 g, 2.87 mmol, 1.1 equiv). The mixture was stirred at 40° C., a clear mixture was obtained after 10 minutes. N-bromosuccinimide (0.47 g, 2.64 mmol, 1.01 equiv) was added in portions. After complete addition the resulting red clear solution was stirred at 40° C. for 1 h. the mixture was diluted with isopropyl acetate (10 ml), yellow-orange suspension was cooled to 0° C. over 1.5 h. After filtration the fine precipitate was solubilised in 50 ml of DCM/MeOH 9/1 v/v. The organic phase was washed with a saturated aqueous solution of NaHCO₃ (25 ml×2), a saturated aqueous solution of NaCl (25 ml×1) and dried over MgSO₄. Solvent was removed under reduced pressure and the product used in next step without purification (0.4 g, 62% yield).

R_{f} 0.73 (DCM/MeOH 9/1); ¹H NMR (500 MHz, DMSO) δ 8.45-8.40 (m, 1H), 7.27-7.17 (m, 3H), 7.09 (dd, J=5.2, 1.7 Hz, 1H), 2.29 (s, 3H), 1.32 (s, 9H). ¹³C NMR (126 MHz, DMSO) δ 168.7, 166.7, 148.7, 146.9, 140.8, 118.8, 116.5, 115.4, 37.1, 29.9, 16.9.

### 6.2. Synthesis of Compound 18

Compound 18 was synthesized by a process as described above in example 4 for the synthesis of compound 5 except that 5-(2-(tert-butyl)pyridin-4-yl)-4-methylthiazol-2-amine, obtained as described hereabove, and intermediate 25 were used as starting materials to obtain compound 18. Characterization is detailed in Table 1b.

Similar processes were performed using compound D and intermediates compounds 23 and 36 to obtain compounds 19 and 20. Characterization of the obtained compounds are given in Table 1b.

### Example 7: PI3K-Inhibitory Activity

Inhibitory activity of compounds against the Human PtdIns (4,5) P 3-kinase activity was quantified employing the enzymatic HTRF (Homogeneous Time Resolved Fluorescence) Assay.

PI3K p110a/p65a (h) is incubated in assay buffer containing 10 μM phosphatidylinositol 4,5-bisphosphate and Mg/ATP (concentration as required). The reaction is initiated by the addition of the ATP solution. After incubation for 30 minutes at room temperature, the reaction is stopped by the addition of stop solution containing EDTA and biotinylated phosphatidylinositol-3,4,5-trisphosphate. Finally, detection buffer is added, which contains europium-labelled anti-GST monoclonal antibody, GST-tagged GRP1 PH domain and streptavidin allophycocyanin. The plate is then read in time resolved fluorescence mode and the homogeneous time-resolved fluorescence (HTRF) signal is determined according to the formula HTRF=10000×(Em665 nm/Em620 nm)

### Results

The tested compounds were classified according to their IC50 values as follows:
A: IC50 activity lower or equal to 50 nM;
B: IC50 strictly greater than 50 nM and up to 200 nM;
C: IC50 strictly greater than 200 nM and up to 500 nM;
D: IC50 strictly greater than 500 nM and up to 1000 nM;
E: IC50 strictly greater than 1 μM

| | Class | Class | Class | Class |
|---|---|---|---|---|
| Cpd No | IC₅₀ PI3K-α | IC₅₀ PI3K-β | IC₅₀ PI3K-γ | IC₅₀ PI3K-δ |
| 1 | C | E | E | C |
| 2 | B | E | C | C |
| 3 | B | E | C | E |
| 4 | E | E | E | E |
| 5 | A | E | C | C |
| 6 | E | E | E | E |
| 7 | B | E | C | D |
| 8 | B | E | C | D |
| 9 | C | E | E | E |
| 10 | E | E | E | E |
| 12 | D | E | E | E |
| 13 | A | E | C | C |
| 14 | A | E | C | C |

It is therefore apparent that the tested compounds of formula (I) in accordance with the invention are potent inhibitors of PI3K kinases, and in particular of PI3K-α kinase. Most compounds further exhibit a selective inhibitory activity against PI3K-α kinase, with respect to PI3K-β and/or PI3K-γ and/or PI3K-δ.

The compounds of formula (I) can therefore be used for preparing medicaments, especially medicaments which can be used in the prevention and/or treatment of Protein tyrosine kinase mediated diseases, in particular PI3K mediated diseases, more particularly of diseases mediated by the isoform alpha of PI3K.

Thus, another aspect of the invention relates to a compound of formula (I) as defined above, or a deuterated or tritiated form of the compound of formula (I), or any of its pharmaceutically acceptable salts, in particular at least any of compounds (1) to (20), for use as a medicament.

In particular, the compounds of formula (I) may be used in the treatment and/or the prevention of pathologies mediated by the activation of the protein tyrosine kinase, in particular of PI3K, especially of PI3K alpha.

According to another of its aspects, the invention thus relates to a compound of formula (I), or a deuterated or tritiated form of the compound of formula (I), or anyone of its pharmaceutically acceptable salts, for use in the treatment and/or prevention of Protein tyrosine kinase mediated diseases, in particular PI3K mediated diseases, more particularly of diseases mediated by PI3K alpha.

Another aspect of the disclosure relates to the use of a compound of formula (I), or a deuterated or tritiated form of the compound of formula (I), or anyone of its pharmaceutically acceptable salts, for treating and/or preventing Protein tyrosine kinase mediated diseases, in particular PI3K mediated diseases, more particularly of diseases mediated by PI3K alpha.

Another aspect of the disclosure relates to the use of a compound of formula (I), or a deuterated or tritiated form of the compound of formula (I), or anyone of its pharmaceutically acceptable salts, for the preparation/manufacture of a composition, such as a medicament, for treating and/or preventing Protein tyrosine kinase mediated diseases, in particular PI3K mediated diseases, more particularly of diseases mediated by PI3K alpha.

Described is also a method for treating and/or preventing Protein tyrosine kinase mediated diseases, in particular PI3K mediated diseases, more particularly of diseases mediated by PI3K alpha.

The methods and uses defined here-above may comprise administering to a subject in need thereof a therapeutically effective amount of a compound of formula (I), or a deuterated or tritiated form of the compound of formula (I), or a pharmaceutically acceptable salt thereof; or a pharmaceutical composition comprising it.

The Protein tyrosine kinase mediated diseases targeted by the uses and methods according to the invention may be more particularly PI3K related Overgrowth Spectrum (PROS), proliferative diseases, in particular cancers, and inflammatory diseases, including autoimmune disorders, as detailed hereafter.

The Protein tyrosine kinase mediated diseases targeted by the uses and methods according to the invention may also more particularly be a mitochondrial genetic disease.

The Protein tyrosine kinase mediated diseases targeted by the uses and methods according to the invention may also more particularly be selected from the group consisting of keloids, hypertrophic scars including burn scars and hyperpigmentation disorders.

More particularly, the Protein tyrosine kinase mediated diseases, in particular phosphatidylinositol 3-kinase mediated diseases, targeted by the uses and methods according to the invention may be selected from the group consisting of PI3K related Overgrowth Spectrum (PROS); neurofibromatosis; mitochondrial genetic diseases; keloids; hypertrophic scars, including burn scars; hyperpigmentation disorders; proliferative diseases, in particular cancers, inflammatory diseases including autoimmune disorders and glomerulonephritis, and more particularly selected from the group consisting of keloids; hypertrophic scars, including burn scars; and hyperpigmentation disorders.

### PI3K Related Overgrowth Spectrum

The phosphatidylinositol 3-kinase related Overgrowth Spectrum is a group of disorders including, but not limited to, fibroadipose overgrowth (FAO), megalencephaly-capillary malformation (MCAP) syndrome, congenital lipomatous asymmetric overgrowth of the trunk, lymphatic, capillary, venous, and combined-type vascular malformations, epidermal nevi, skeletal and spinal anomalies (CLOVES) syndrome, Hemihyperplasia Multiple Lipomatosis (HHML), Klippel-Trenaunay syndrome, isolated and complex venous malformations, isolated and complex lymphatic malformations, or combined complex vascular malformations (see for example Venot Q. et al., Nature 2018 June; 558 (7711): 540-546; Canaud G. et al., Orphanet J Rare Dis. 2021 Jul. 8; 16 (1): 306; Morin G, Canaud G., Br Med Bull. 2021 Dec. 16; 140 (1): 36-49; Delestre F. et al., Sci Transl Med. 2021. Oct. 6; 13 (614); and Morin G. et al. J Exp Med. 2022 Mar. 7; 219 (3): e20212148).

The fibroadipose overgrowth (FAO) is a syndrome characterized by the major findings of segmental progressive overgrowth of subcutaneous, muscular, and visceral fibroadipose tissue with skeletal overgrowth.

The megalencephaly-capillary malformation (MCAP) syndrome is characterized by the major findings of (1) megalencephaly (MEG) or hemimegalencephaly (HMEG) associated with neurologic findings of hypotonia, seizures, and mild to severe intellectual disability; and (2) cutaneous capillary malformations with focal or generalized somatic overgrowth.

CLOVES relates to Congenital, Lipomatous, Overgrowth, Vascular Malformations, Epidermal Nevi and Spinal/Skeletal Anomalies and/or Scoliosis. This syndrome is characterised by lipomatous tissues showing complex congenital overgrowth (typically appearing as a truncal lipomatous mass) and a combination of vascular and lymphatic malformations.

The Hemihyperplasia Multiple Lipomatosis (HHML) is a condition characterized by asymmetric nonprogressive overgrowth, multiple lipomas, and superficial vascular malformations.

A Klippel-Trenaunay syndrome is a rare congenital medical condition in which blood vessels and/or lymph vessels fail to form properly.

According to an aspect, the patients to be treated may have a PIK3CA mutation, in particular a PIK3CA mutation selected from the group comprising mutation H1047R, mutation C420R, mutation H1047L, mutation E542K, mutation E545K and/or mutation Q546R.

### Neurofibromatosis

Neurofibromatosis refers to a rare genetic disorder that causes typically benign tumors on or just under the skin and also in nerves near the spinal cord or along nerves elsewhere in the body. There are three types of Neurofibromatosis (NF): neurofibromatosis type 1 (NF1), neurofibromatosis type 2 (NF2), and schwannomatosis. The neurofibromatosis type 1 (NF1) is caused by mutations in the NF1 gene that lead to the production of a nonfunctional version of neurofibromin that cannot regulate cell growth and division. As a result, tumors such as neurofibromas can form along nerves throughout the body. NF1 is an autosomal dominant disorder. The neurofibromatosis type 2 (NF2) is caused by mutations in the NF2 gene cause neurofibromatosis type 2. NF2 also known as MISME syndrome for multiple inherited schwannomas, meningiomas, and ependymomas. Signs and symptoms of NF2 usually result from the development of benign, slow-growing tumors (acoustic neuromas) in both ears. Also known as vestibular schwannomas, these tumors grow on the nerve that carries sound and balance information from the inner ear to the brain. Schwannomatosis causes tumors to develop on skull (cranial), spinal and peripheral nerves but not on the nerve that carries sound and balance information from the inner ear to the brain. Concerning neurofibromatosis, reference can be made to the PCT application WO2020053125.

### Keloids, Hypertrophic Scars and Hyperpigmentation Disorders

Concerning the use of PI3K inhibitors in the treatment these disorders, reference can for example be made to the PCT application WO2020201073. Reference can also be made to Mari W. et al. (J Am Coll Clin Wound Spec. 2016 Nov. 30; 7 (1-3): 1-7).

Usually scars are well delimited. However, in some conditions, fibroblasts and myofibroblasts overproduce collagen (type I and III) leading to hypertrophic scars. These hypertrophic scars are restricted to the original wound area. Importantly, under certain circumstances the scars can grow outside from the original injured area, invading adjacent dermal tissue due to extensive production of extracellular matrix, especially collagen (type I and III), which caused by over expression of cytokines and growth factors. These scars are called keloid.

As used herein the term “keloid” refers to an excessive accumulation of extracellular matrix proteins, leading to an overabundance of collagen formation. Abnormal skin scarring can occur, post-injury in genetically susceptible individuals. As used herein, the terms “keloid scars” refer to an excessive scar in which the dense fibrous tissue extends beyond the borders of the original wound or incision and does not usually regress spontaneously.

As used herein the terms “hypertrophic scars” refer to an overgrowth of dense fibrous tissue that result from abnormal wound healing. In contrast to keloids, hypertrophic scars do not extend beyond the original boundaries of a wound. Also, unlike keloids, hypertrophic scars typically reach a certain size and then stabilize or regress. Hypertrophic scars include hypertrophic burn scars, which is the most common complication of a burn injury.

As used herein the terms “hyperpigmentation disorders” or “hyperpigmentary skin disorder” are used interchangeably and refer to the darkening of an area of skin or nails caused by increased melanin. Hyperpigmentation is the result of either of two occurrences: (1) an abnormally high concentration of melanocytes produces melanin or (2) when melanocytes are hyperactive. Hyperpigmentation disorders can affect any part of the body including the face, hands, and neck. Hyperpigmentation disorder is selected from the group comprising, and in particular consisting of, solar lentigines, melasma, freckles, age spots, post-acne pigmentation and post-inflammatory hyperpigmentation.

The terms “lentigo/lentigenes” or “solar lentigines,” also known as a sun-induced freckle or senile lentigo, are a dark (hyperpigmented) lesion caused by natural or artificial ultraviolet (UV) light. The term “melasma” is also known as pregnancy-induced melasma. It is also known as pregnancy mask or chloasma. With melasma, the pigmentation is generally symmetrical and has clearly defined edges. The term “freckles” refers to flat circular spots which are usually tan or light brown in color. While freckles are an extremely common type of hyperpigmentation, they are more often seen among people with a lighter skin tone. The term “age spots” refers to tan, brown or black in color. Age spots are oval in shape and the size varies from freckle size to more than 13 mm. It is also known as liver spots and they tend to develop on the face and other photo-exposed areas after the age of 40. The terms “post acne pigmentation” refer to marks caused by acne. They can be observed in more than 60% of acne in some ethnics. In most cases pigmentary marks which are dark in color result from an overproduction of melanin in reaction to skin inflammation at the affected area. Without proper treatment, post-acne pigmentation may take months or even years to fade off. The term “post inflammatory hyperpigmentation” refers to the marks caused by an injury or inflammation to the skin, there is an increased production of color pigment in such conditions.

### Cancers

A compound of formula (I), or a deuterated or tritiated form of the compound of formula (I), or pharmaceutically acceptable salts thereof, as defined above, may be useful in the treatment or prevention of various cancers.

In particular, the compounds of formula (I) may be used as anticancer agents, in particular for use in the treatment of the cancers detailed hereafter.

Among cancers, the following may be cited: Blood-Related Cancer, pancreatic cancer, urological cancer, bladder cancer, colorectal cancer, colon cancer, breast cancer, prostate cancer, renal cancer, hepatocellular cancer, thyroid cancer, gall bladder cancer, kidney cancer, liver cancer, lung cancer, such as non-small cell lung cancer and small-cell lung cancer, ovarian cancer, cervical cancer, gastric cancer, endometrial cancer, oesophageal cancer, head and neck cancer, such as head and neck squamous cell carcinoma (HNSCC), melanoma, neuroendocrine cancer, CNS cancer, brain cancer, such as glioma, glioblastoma, anaplastic oligodendroglioma, and anaplastic astrocytoma, bone cancer, hematological cancer, such as leukemia, lymphoma and myeloma, soft tissue sarcoma, retinoblastomas, neuroblastomas, peritoneal effusions, malignant pleural effusions, mesotheliomas, Wilms tumors, trophoblastic neoplasms, hemangiopericytomas, Kaposi's sarcomas, myxoid carcinoma, round cell carcinoma, squamous cell carcinomas, oesophageal squamous cell carcinomas, oral carcinomas, cancers of the adrenal cortex, or ACTH-producing tumors.

According to one embodiment, the following cancers may be cited: bladder cancer, breast cancer, lung cancer, such as non-small cell lung cancer and small-cell lung cancer, ovarian cancer, cervical cancer, kidney cancer, liver cancer, head and neck cancer, such as head and neck squamous cell carcinoma (HNSCC), sarcoma, brain cancer, such as glioma, glioblastoma, anaplastic oligodendroglioma and anaplastic astrocytoma, or hematological cancer, such as leukemia, lymphoma and myeloma.

According to another embodiment, the cancer may be selected from head and neck cancer, Head and Neck Squamous Cell Carcinoma, Neck Squamous Cell Carcinoma, Acute Lymphocytic Leukemia (ALL) in Adults or children, Acute Myeloid Leukemia (AML) in adults or children, Acute Lymphoblastic Leukemia, Astrocytic Glioma, B- or NK/T-cell lymphomas, Bile Duct Cancer, Bladder Cancer, Brain and Spinal Cord Tumors in Adults, Brain and Spinal Cord Tumors in Children, Anaplastic astrocytomas, Breast Cancer in Women, Breast Cancer in Young Women, Breast Cancer in Men, Recurrent Breast Cancer, Hereditary Breast Cancer, HER2 positive Breast Cancer, Breast Cancer associated with lymph node metastatis, ER-alpha positive Breast Cancer, Chronic Lymphocytic Leukemia (CLL), Chronic Myeloid Leukemia (CML), Chronic Myelomonocytic Leukemia (CMML), Epithelial Ovarian Cancer, Epithelial Ovarian Cancer associated with metastasis, Ewing sarcoma, Ewing Family of Tumors, Lymphoblastic leukaemia (ALL), Eye Cancer, such as Ocular Melanoma and Lymphoma, Gestational Trophoblastic Disease, Glioblastoma, Glioblastoma multiforme (GBM), Hairy cell leukemia, Glioma, High-grade glioma, Hepatocellular carcinoma, Intrahepatic cholangiocarcinoma, Invasive Breast Ductal Carcinoma, Hodgkin Lymphoma, Kaposi Sarcoma, Leiomyosarcoma, Leukemia, Leukemia in Children, Lung Carcinoid Tumor, Lymphoma, Lymphoma of the Skin, Mantle cell lymphoma, Medulloblastoma, Melanoma Skin Cancer, malignant melanoma, Neuroblastoma, Neuroglioma, Non-Hodgkin Lymphoma, Non-Hodgkin Lymphoma in Children, Non-Small Cell Lung Cancer, Gefitinib-resistant non-small cell lung cancer, Osteosarcoma, Pulmonary Metastatic Osteosarcoma or lymphoproliferation.

Lymphoproliferation, or lymphoproliferative disorders (LPD), refers to a heterogeneous group of diseases characterized by uncontrolled production of lymphocytes that cause monoclonal lymphocytosis, lymphadenopathy and bone marrow infiltration. These diseases often occur in immunocompromised individuals. There are two subsets of lymphocytes: T and B cells that regenerate uncontrollably to produce immunoproliferative disorders, which are prone to immunodeficiency, a dysfunctional immune system, and lymphocyte dysregulation.

In a particular embodiment, the lymphoproliferative disorder is a B-cell lymphoproliferative disorder.

In a particular embodiment, the B-lymphoproliferative disorder is selected from the group consisting of but not limited to: Hodgkin's lymphoma, Diffuse large B-cell lymphoma, acute lymphocytic leukemia, lymphoid blastic phase Chrome Myeloid Leukemia, Chronic lymphocytic leukemia/Small lymphocytic lymphoma, Extranodal marginal zone B-cell lymphomas, Mucosa-associated lymphoid tissue lymphomas, Follicular lymphoma, Mantle cell lymphoma, Nodal marginal zone B-cell lymphoma, Burkitt lymphoma, Hairy cell leukemia, Primary central nervous system lymphoma, Splenic marginal zone B-cell lymphoma, Waldenstrom's macroglobulinemia/Lymphoplasmacytic lymphoma, Multiple 20 myeloma, Plasma cells dyscrasias, Plasma cell neoplasms, Primary mediastinal B-cell lymphoma, Hodgkin Disease or Castelman's Disease.

In a particular embodiment, the lymphoproliferative disorder is a T-cell lymphoproliferative disorder.

In a particular embodiment, the T-lymphoproliferative disorder is selected from the group consisting of but not limited to: leukemia/lymphoma, Extranodal natural killer/T-cell lymphoma, Cutaneous T-cell lymphoma, Enteropathy-type T-cell lymphoma, Angioimmunoblastic T-cell lymphoma, Anaplastic large T/null-cell lymphoma, Subcutaneous panniculitis-like T-cell lymphoma, T-cell acute lymphocytic leukemia, T-cell large granular lymphocyte leukemia, Lymphoid blastic phase Chrome Myeloid Leukemia, post transplantation lymphoproliferative syndromes, human T-cell leukemia virus type 1-positive (HTLV-G) adult T-cell leukemia/lymphoma (ATL), T-cell prolymphocytic leukemia (TPLL), or unspecified T-cell lymphoma.

According to one embodiment, the patient does not present clinically detectable metastases, in particular said patient has a pre-cancerous condition, an early stage cancer or a non-metastatic cancer, or said patient presents clinically detectable metastases and said compound of formula (I), or a deuterated or tritiated form of the compound of formula (I), or anyone of its pharmaceutically acceptable salts as defined above do not target directly the invasion of metastases.

### Glomerulonephritis

Glomerulonephritis is a group of renal diseases characterized by immune-mediated damage to the basement membrane, mesangium, or the capillary endothelium, leading to hematuria, proteinuria, and azotemia involving damages to the glomeruli.

Glomerulonephritis according to the invention may in particular be proliferative or non-proliferative.

Non-proliferative glomerulonephritis is characterised by a lack of glomerular cell proliferation and typically presents with nephrotic syndrome.

Proliferative glomerulonephritis (PGN) refers to an increase of cellularity of the glomerulus, which due to proliferation of intrinsic glomerular cells, infiltration of leucocytes, or both. This principally occurs in the context of glomerular deposition of immunoglobulins, immune complexes, or complement components. Different subtypes are described based on histological features: proliferation of mesangial cells, endocapillary proliferation, diffuse proliferation, or extracapillary proliferation (also termed crescentic glomerulonephritis).

The proliferative glomerulonephritis may be caused by the following diseases selected from the group consisting of but not limited to: Infectious disease (poststreptococcal glomerulonephritis, infective endocarditis, occult visceral sepsis, hepatitis B infection—with vasculitis and/or cryoglobulinemia—, HIV infection, hepatitis C with cryoglobulinemia, membranoproliferative glomerulonephritis-), multisysteme diseases (systemic lupus erythematosus, IgA nephropathy, Henoch-Schönlein purpura, systemic necrotizing vaculitis—including granulomatosis with polyangtiitis type Wegener, Goodpasture's syndrome, essential mixed cryoglobulinemia, malignancy, relapsing polychondritis, rheumatoid arthritis—with vasculitis).

In a particular embodiment, the proliferative glomerulonephritis is caused by systemic lupus erythematosus. As used herein, the term “systemic lupus erythematosuisus” (SLE) refers to a systemic autoimmune disease thought to be manifested by a wide range of abnormalities in immune regulation. It is the most common type of lupus.

The proliferative glomerulonephritis may be lupus nephritis.

As used herein, the term “lupus nephritis” (LN) refers to an inflammation of the kidney that is caused by systemic lupus erythematosus (SLE). Up to 60% of lupus patients will develop LN. When the kidneys are inflamed, they cannot function normally to filter toxins, byproducts, excess salts, excess fluid, and other impurities the blood. If not controlled, LN can lead to kidney failure. Even with treatment, loss of kidney function sometimes progresses. If both kidneys fail, subjects with LN may need dialysis. Ultimately, it may be necessary for the LN subject to receive a kidney transplant. Symptoms of loss of or abnormal kidney function include increased amounts of protein in urine (proteinuria), foaming in a subject's urine, and/or a higher level of blood urea nitrogen (BUN).

In a particular embodiment, the subject is a human afflicted with or susceptible to be afflicted with infectious disease (poststreptococcal glomerulonephritis, infective endocarditis, occult visceral sepsis, hepatitis B infection—with vasculitis and/or cryoglobulinemia—, HIV infection, hepatitis C—with cryoglobulinemia, membranoproliferative glomerulonephritis—) or multisysteme diseases (systemic lupus erythematosus, IgA nephropathy, Henoch-Schönlein purpura, systemic necrotizing vaculitis—including granulomatosis with polyangtiitis type Wegener, Goodpasture's syndrome, essential mixed cryoglobulinemia, malignancy, relapsing polychondritis, rheumatoid arthritis—with vasculitis).

### Mitochondrial Genetic Diseases

Mitochondrial genetic diseases refer to a clinically and genetically heterogeneous group of disorders that arise as a result of mitochondrial dysfunction. Mitochondrial genetic disorders are caused by mutations in either the mitochondrial DNA or nuclear DNA that lead to dysfunction of the mitochondria and inadequate production of energy. Mitochondrial genetic disorders may in particular be selected from the group consisting of mitochondrial cytopathy; aminoglycoside induced deafness; chronic progressive external ophthalmoplegia; depletion syndromes; Kearns-Sayre syndrome; Leber's hereditary optic neuropathy; Leigh syndrome; Cerebellar Hypoplasia, Mitochondrial myopathy Encephalopathy Lactic Acidosis, and Stroke-like episodes (MELAS); myoclonic epilepsy and ragged red fibres; maternally inherited Leigh syndrome; neurogenic weakness, ataxia, and retinitis pigmentosa; Pearson syndrome; microcephaly, optic atrophy, lactic acidosis, Optic nerve atrophy, Spastic paraplegia, Friedreich's ataxia, Sideroblastic anaemia and ataxia, Sideroblastic anaemia, Encephalomyopathy, tubulopathy, ataxia, Hypertrophic cardiomyopathy LS and Alpers syndrome.

A compound of formula (I) according to the invention, or a deuterated or tritiated form of the compound of formula (I), or a pharmaceutically acceptable salt thereof, may particularly be suitable for use in the treatment and/or prevention of phosphatidylinositol 3-kinase related Overgrowth Spectrum (PROS), in particular Congenital PROS, Lipomatous, Overgrowth, Vascular Malformations, Epidermal Nevi and Spinal/Skeletal Anomalies and/or Scoliosis (CLOVES) syndrome.

A compound of formula (I) according to the invention, or a deuterated or tritiated form of the compound of formula (I), or a pharmaceutically acceptable salt thereof, may particularly be suitable for use in the treatment and/or prevention of cancer, such as the here above listed cancers.

Described herein is also the use of a compound of formula (I), or a deuterated or tritiated form of the compound of formula (I), or a pharmaceutically acceptable salt thereof, for treatment and/or prevention of cancer, such as the here above listed cancers.

The compounds of the formula (I), or a deuterated or tritiated form of the compounds of formula (I), or pharmaceutically acceptable salt thereof, may be used in monotherapy or combination with another therapy selected from chemotherapy, immunotherapy, radiotherapy, surgery, ultrasounds, monoclonal antibodies, anti-tumoral vaccines, RNA vaccines, cancer vaccines, magnetic particles, intravascular microrobots.

Therefore, another aspect of the disclosure is a compound of formula (I), or a deuterated or tritiated form of the compound of formula (I), or a pharmaceutically acceptable salt thereof, for use as an antitumor agent intended for patients who are also treated with anyone of chemotherapy, immunotherapy, radiotherapy, surgery, ultrasounds, monoclonal antibodies, anti-tumoral vaccines, RNA vaccines, cancer vaccines, magnetic particles, intravascular microrobots.

According to another of its aspects, the present invention relates to a medicament that comprises a compound of formula (I), or a deuterated or tritiated form of the compound of formula (I), or any of its pharmaceutically acceptable salts.

The medicaments may for example be employed therapeutically in the treatment and/or prevention of the Protein tyrosine kinase mediated diseases, in particular PI3K mediated diseases, more particularly of diseases mediated by PI3K alpha, and especially of the diseases and conditions detailed hereabove.

According to another of its aspects, the present invention relates to pharmaceutical compositions comprising at least one compound of formula (I) as defined above, or a deuterated or tritiated form of the compound of formula (I), or a pharmaceutically acceptable salt thereof, in particular at least one of compounds (1) to (20). These pharmaceutical compositions contain in particular an effective dose of at least one compound of formula (I), or a deuterated or tritiated form of the compound of formula (I), or a pharmaceutically acceptable salt thereof.

A pharmaceutical composition according to the present invention can contain one or more compound(s) of the invention in any form described herein.

The compounds can be administered through any mode of administration such as, for example, topical, intramuscular, intravenous, intranasal, subcutaneous or oral route, as a suppository, etc.

In one embodiment, a pharmaceutical composition according to the present invention is selected from an oral composition; a topical composition; an inhalation composition; an injectable composition, in particular a sub-cutaneous composition, an intramuscular composition or an intravenous composition, a suppository, and an oral, injectable or surgical sustained release composition.

The pharmaceutical compositions of the invention may also contain at least one pharmaceutically acceptable excipient.

The said excipients are chosen, according to the pharmaceutical form and the desired mode of administration, from the usual excipients known to those skilled in the art. They may be chosen among carriers, glidants, diluents, excipients, stabilizers and preservatives. Such additives are well known to those skilled in the art and are described notably in “Ullmann's Encyclopedia of Industrial Chemistry, 6^{th} Ed.” (various editors, 1989- 1998, Marcel Dekker) and in “Pharmaceutical Dosage Forms and Drug Delivery Systems” (ANSEL et al., 1994, WILLIAMS & WILKINS).

The compounds of the invention may be used in monotherapy or combination with radiotherapy or chemotherapy. In an embodiment, pharmaceutical compositions of the invention may further comprise at least another chemotherapeutic agent.

### Example 8: Compounds of the Invention Improve Kidney Lesions in Lupus Model

Compound 5 of the invention was tested in NZBWF1/J mice, a well-established model that exhibits lupus-like nephritis (Celhar, T. & Fairhurst, A. M. Modelling clinical systemic lupus erythematosus: similarities, differences and success stories. Rheumatology (Oxford) 56, 188-199 (2017)). NZBWF1/J mice gradually develop immune glomerulonephritis characterized by proteinuria and kidney dysfunction starting around 25 weeks of age. The uninephrectomy model was employed and uninephrectomy was performed on 12 female mice at 24 weeks of age.

As the incidence and severity of symptoms are more pronounced in females, only females were used in this study. The mice were then randomly assigned to receive either the vehicle (n=6) or Compound 5 50 mg/kg/day orally (n=6) for a duration of 4 weeks. At the end of the treatment period, the mice were sacrificed and their kidney histology was compared to samples obtained during the uninephrectomy procedure.

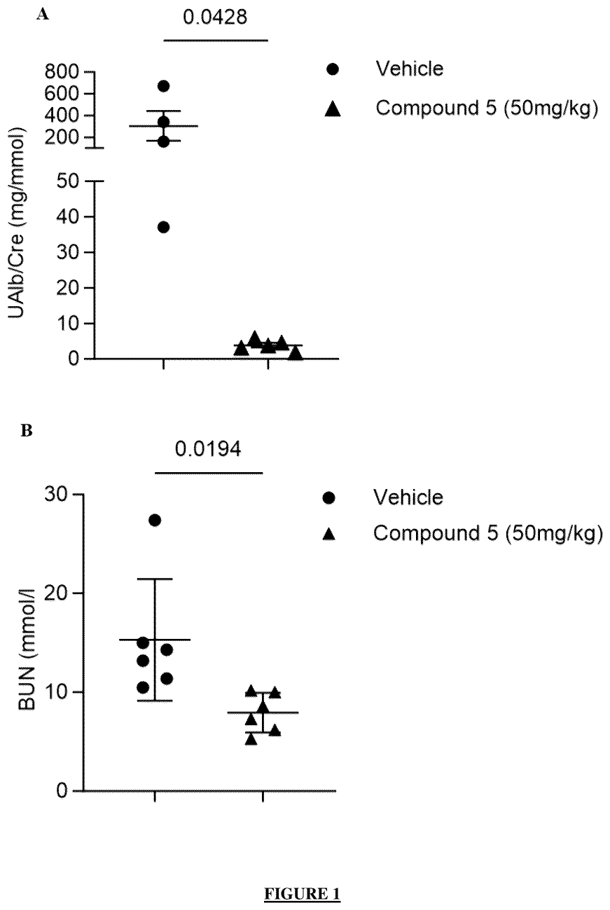

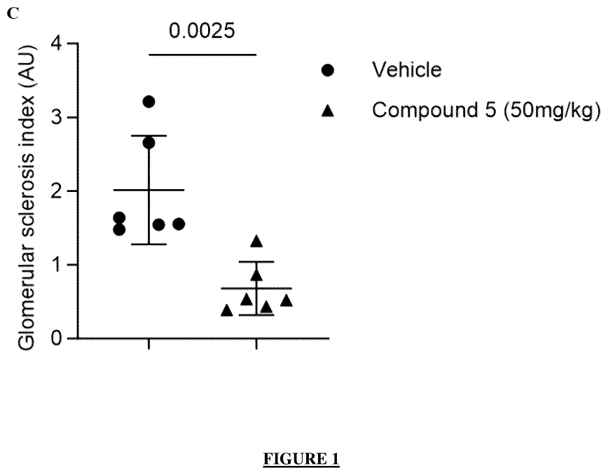

At the time of uninephrectomy, no discernible differences were observed between the two groups in terms of phenotypic characteristics including proteinuria and the kidney-to-body weight ratio. However, upon sacrifice, mice treated with Compound 5 exhibited significant reductions in albuminuria (FIG. 1A) and blood urea nitrogen levels (FIG. 1B).

Furthermore, the kidney-to-body weight ratio was notably decreased in uninephrectomized NZBWF1/J mice receiving Compound 5. From a histological standpoint, the Compound 5-treated mice displayed preserved glomeruli compared to those receiving the vehicle (FIG. 1C). While glomerular lesions worsened considerably in the vehicle-treated uninephrectomized NZBWF1/J mice, they remained stable in the Compound 5 group.

Collectively, these findings indicate that a compound according to the invention attenuates glomerulonephritis lesions and particularly lupus nephritis.

### Example 9: Compounds of the Invention Induce a Low Increase in Blood Glucose

C57B16 mice (n=6 mice per group, 3 males and 3 females) aged of 8 weeks old were daily treated by either vehicle or Compound 5 (50 mg/kg) during 5 consecutive days through oral gavage.

On day 6, blood glucose concentrations were measured at different time points before and after drug administration.

Whereas compounds currently available in the market are known for being associated with a peak of glycemia around 2 hours after oral gavage, Compound 5 only induced a modest increase in blood glucose. Ordinary one way Anova multiple comparison: significantly significative at 2 hours.

### Example 10: Compounds of the Invention do not Increase Blood Insulin Concentration

C57B16 mice (n=3 mice per group, 3 males) aged of 8 weeks old were daily treated by either vehicle or Compound 5 (50 mg/kg) during 5 consecutive days through oral gavage. On day 6, insulin concentrations were measured at different time points before and after drug administration.

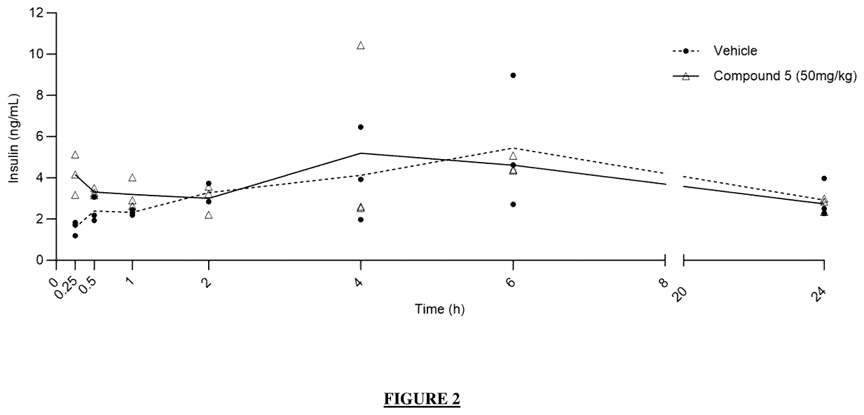

Insulin level was measured in plasma (7 μL in duplicate) using MSD u.plex (Mesoscale, ref 1526HK).

Whereas compounds currently available in the market are known for being associated with a peak of insulin around 4 hours after oral gavage which is persistent up to 8 hours after drug administration, Compound 5 induced no increase in blood insulin concentration (FIG. 2).

1-14. (canceled)

15. A compound of formula (I), or a deuterated or tritiated form of the compound of formula (I), or a pharmaceutically acceptable salt thereof: 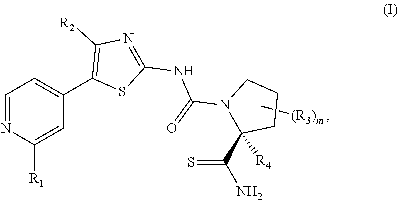
wherein:
R₁ is a (C₁-C₆)alkyl group or a (C₃-C₆) cycloalkyl group, unsubstituted or substituted with one or more fluorine atoms;
R₂ is chosen from:
a hydrogen atom, and
a (C₁-C₆)alkyl group,
m is 0, 1 or 2;
each R₃, when present, is independently chosen from:
a fluorine atom,
a (C₁-C₆)alkyl group, unsubstituted or substituted with one or more halogen atoms,
a (C₃-C₆) cycloalkyl group unsubstituted or substituted with one or more halogen atoms,
a hydroxy group,
a (C₁-C₆)alkoxy group, and
a —NRR′ group, R and R′ being independently chosen from a hydrogen atom and a (C₁-C₆)alkyl group,
or two R₃, that are borne by the same carbon atom, form with the carbon atom bearing them a (C₃-C₆) cycloalkyl ring, unsubstituted or substituted with one or more fluorine atoms; and
R₄ is chosen from:
a fluorine atom,
a hydrogen atom,
a (C₁-C₆)alkyl group, unsubstituted or substituted with one or more halogen atoms, and
a (C₃-C₆) cycloalkyl group, unsubstituted or substituted with one or more halogen atoms,
or R₃ and R₄, when they are borne by two adjacent carbon atoms, form with the carbon atoms bearing them a (C₃-C₆) cycloalkyl ring, unsubstituted or substituted with one or more halogen atom.

16. The compound of formula (I) according to claim 15, or a deuterated or tritiated form of the compound of formula (I), or a pharmaceutically acceptable salt thereof, wherein R₁ is a (C₁-C₆)alkyl group, in particular a (C₁-C₄)alkyl group, and more particularly a tert-butyl group, unsubstituted or substituted with one or more fluorine atoms.

17. The compound of formula (I) according to claim 15, or a deuterated or tritiated form of the compound of formula (I), or a pharmaceutically acceptable salt thereof, wherein R₂ is a (C₁-C₆)alkyl group, in particular a (C₁-C₄)alkyl group and more particularly a methyl group.

18. The compound of formula (I) according to claim 15, or a deuterated or tritiated form of the compound of formula (I), or a pharmaceutically acceptable salt thereof, wherein m is 1 or 2, and each R₃ is independently chosen from:
a fluorine atom,
a (C₁-C₆)alkyl group, in particular a (C₁-C₄)alkyl group and more particularly a methyl group, unsubstituted or substituted with one or more halogen atoms; for example a methyl group or a trifluoromethyl group;
a hydroxy group, and
a (C₁-C₆)alkoxy group, in particular a (C₁-C₄)alkoxy group and more particularly a methoxy group;
or, wherein m is 2, and two R₃, that are borne by the same carbon atom, form with the carbon atom bearing them a (C₃-C₆) cycloalkyl ring, in particular a cyclopropyl ring, unsubstituted or substituted with one or more fluorine atoms.

19. The compound of formula (I) according to claim 15, or a deuterated or tritiated form of the compound of formula (I), or a pharmaceutically acceptable salt thereof, wherein R₄ is chosen from:
a hydrogen atom, and
a (C₁-C₆)alkyl group, in particular a (C₁-C₄)alkyl group and more particularly a methyl group, unsubstituted or substituted with one or more halogen atoms, in particular with one or more fluorine atom.

20. The compound of formula (I) according to claim 15, or a deuterated or tritiated form of the compound of formula (I), or a pharmaceutically acceptable salt thereof, said compound being selected from the following compounds:
(1) (S)-2-carbamothioyl-4,4-difluoro-N-(4-methyl-5-(2-(1,1,1-trifluoro-2-methylpropan-2-yl)pyridin-4-yl)thiazol-2-yl)pyrrolidine-1-carboxamide,
(2) (2S,4R)-2-carbamothioyl-4-fluoro-N-(4-methyl-5-(2-(1,1,1-trifluoro-2-methylpropan-2-yl)pyridin-4-yl)thiazol-2-yl)pyrrolidine-1-carboxamide,
(3) (2S,4S)-2-carbamothioyl-4-fluoro-N-(4-methyl-5-(2-(1,1,1-trifluoro-2-methylpropan-2-yl)pyridin-4-yl)thiazol-2-yl)pyrrolidine-1-carboxamide,
(4) (S)-6-carbamothioyl-N-(4-methyl-5-(2-(1,1,1-trifluoro-2-methylpropan-2-yl)pyridin-4-yl)thiazol-2-yl)-1,1-difluoro-5-azaspiro[2,4]heptane-5-carboxamide,
(5) (S)-2-carbamothioyl-N-(4-methyl-5-(2-(1,1,1-trifluoro-2-methylpropan-2-yl)pyridin-4-yl)thiazol-2-yl)pyrrolidine-1-carboxamide,
(6) (S)-2-carbamothioyl-2-methyl-N-(4-methyl-5-(2-(1,1,1-trifluoro-2-methylpropan-2-yl)pyridin-4-yl)thiazol-2-yl)pyrrolidine-1-carboxamide,
(7) (2S,4R)-2-carbamothioyl-4-methoxy-N-(4-methyl-5-(2-(1,1,1-trifluoro-2-methylpropan-2-yl)pyridin-4-yl)thiazol-2-yl)pyrrolidine-1-carboxamide,
(8) (2S,4S)-2-carbamothioyl-4-methoxy-N-(4-methyl-5-(2-(1,1,1-trifluoro-2-methylpropan-2-yl)pyridin-4-yl)thiazol-2-yl)pyrrolidine-1-carboxamide,
(9) (2S,5R)-2-carbamothioyl-5-methyl-N-(4-methyl-5-(2-(1,1,1-trifluoro-2-methylpropan-2-yl)pyridin-4-yl)thiazol-2-yl)pyrrolidine-1-carboxamide,
(10) (2S,5S)-2-carbamothioyl-5-methyl-N-(4-methyl-5-(2-(1,1,1-trifluoro-2-methylpropan-2-yl)pyridin-4-yl)thiazol-2-yl)pyrrolidine-1-carboxamide,
(11) (2S,4S)-2-carbamothioyl-4-methyl-N-(4-methyl-5-(2-(1,1,1-trifluoro-2-methylpropan-2-yl)pyridin-4-yl)thiazol-2-yl)pyrrolidine-1-carboxamide,
(12) (2S,4S)-2-carbamothioyl-N-(4-methyl-5-(2-(1,1,1-trifluoro-2-methylpropan-2-yl)pyridin-4-yl)thiazol-2-yl)-4-(trifluoromethyl)pyrrolidine-1-carboxamide,
(13) (2S,4R)-2-carbamothioyl-4-hydroxy-N-(4-methyl-5-(2-(1,1,1-trifluoro-2-methylpropan-2-yl)pyridin-4-yl)thiazol-2-yl)pyrrolidine-1-carboxamide,
(14) (2S,3S)-2-carbamothioyl-3-hydroxy-N-(4-methyl-5-(2-(1,1,1-trifluoro-2-methylpropan-2-yl)pyridin-4-yl)thiazol-2-yl)pyrrolidine-1-carboxamide,
(15) (2S,3S)-2-carbamothioyl-3-methoxy-N-(4-methyl-5-(2-(1,1,1-trifluoro-2-methylpropan-2-yl)pyridin-4-yl)thiazol-2-yl)pyrrolidine-1-carboxamide,
(16) (S)-6-carbamothioyl-N-(4-methyl-5-(2-(1,1,1-trifluoro-2-methylpropan-2-yl)pyridin-4-yl)thiazol-2-yl)-5-azaspiro[2.4]heptane-5-carboxamide,
(17) (2S,4S)-2-carbamothioyl-4-hydroxy-N-(4-methyl-5-(2-(1,1,1-trifluoro-2-methylpropan-2-yl)pyridin-4-yl)thiazol-2-yl)pyrrolidine-1-carboxamide;
(18) (S)-2-carbamothioyl-N-(4-methyl-5-(2-tert-butyl-pyridin-4-yl)thiazol-2-yl)pyrrolidine-1-carboxamide,
(19) (2S,4S)-2-carbamothioyl-4-fluoro-N-(4-methyl-5-(2-tert-butyl-pyridin-4-yl)thiazol-2-yl)pyrrolidine-1-carboxamide, and
(20) (S)-6-carbamothioyl-N-(4-methyl-5-(2-tert-butyl-pyridin-4-yl)thiazol-2-yl)-5-azaspiro[2.4]heptane-5-carboxamide.

21. A process for preparing a compound of formula (I) as defined in any one of claim 15, or a deuterated or tritiated form of the compound of formula (I), or a pharmaceutically acceptable salt thereof, comprising a coupling reaction between a compound of formula (II-A) and a compound of formula (II-B) 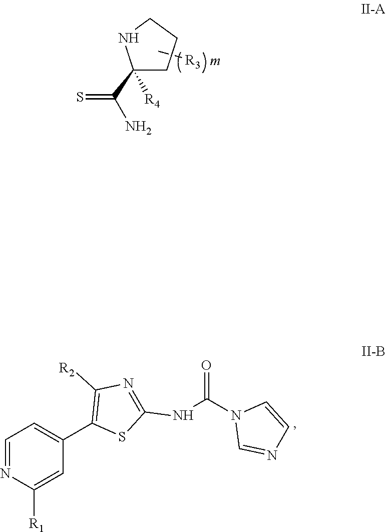
in which R₃, R₄, m, R₂ and R₁ are defined above, provided that, when R₃ is an hydroxy group, R₃ is protected in compound II-A before the coupling reaction, for example with a tert-butyldiphenylsilyl ether (tBDPS) protecting group; the deprotection being performed after the coupling reaction;
wherein said coupling reaction is preferably performed at room temperature using triethylamine in polar aprotic organic solvent such as dimethylformamide;
said coupling reaction being optionally preceded by a step for obtaining compound II-A wherein a compound of formula (I-A) 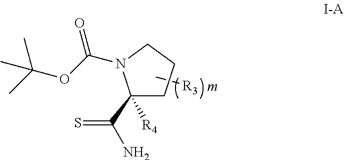
wherein R₃, R₄ and m are as defined above, or R₃ is a protected hydroxy group, for example a hydroxy group protected with a tert-butyldiphenylsilyl ether (tBDPS) protecting group, is converted to a compound II-A by a deprotection step, for example by an aqueous solution of HCl;
said coupling reaction being optionally preceded by a step for obtaining compound II-B wherein a compound of formula (I-B) 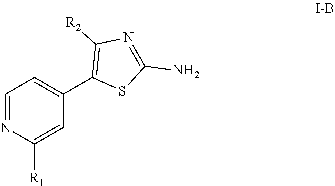
wherein R₁ and R₂ are as defined above, is converted to compound II-B by reaction with 1,1′-carbonyldiimidazole, in particular in an organic solvent, such as methylene chloride.

22. A compound selected from the following compounds:
(21) tert-butyl(S)-2-carbamothioyl-4,4-difluoropyrrolidine-1-carboxylate;
(23) tert-butyl(2S,4S)-2-carbamothioyl-4-fluoropyrrolidine-1-carboxylate,
(24) tert-butyl (6S)-6-carbamothioyl-1,1-difluoro-5-azaspiro[2.4]heptane-5-carboxylate,
(26) tert-butyl(S)-2-carbamothioyl-2-methylpyrrolidine-1-carboxylate,
(28) tert-butyl(2S,4S)-2-carbamothioyl-4-methoxypyrrolidine-1-carboxylate,
(29) tert-butyl(2S,5R)-2-carbamothioyl-5-methylpyrrolidine-1-carboxylate,
(30) tert-butyl(2S,5S)-2-carbamothioyl-5-methylpyrrolidine-1-carboxylate,
(31) tert-butyl(2S,4S)-2-carbamothioyl-4-methylpyrrolidine-1-carboxylate,
(32) tert-butyl(2S,4S)-2-carbamothioyl-4-(trifluoromethyl)pyrrolidine-1-carboxylate,
(34) tert-butyl(2S,3S)-3-((tert-butyldiphenylsilyl)oxy)-2-carbamothioylpyrrolidine-1-carboxylate,
(35) tert-butyl(2S,3S)-2-carbamothioyl-3-methoxypyrrolidine-1-carboxylate,
(36) tert-butyl(S)-6-carbamothioyl-5-azaspiro[2.4]heptane-5-carboxylate, and
(37) tert-butyl(2S,4S)-4-((tert-butyldiphenylsilyl)oxy)-2-carbamothioylpyrrolidine-1-carboxylate;
or a deuterated or tritiated form of the compound of formula (I), or pharmaceutically acceptable salts thereof.

23. A pharmaceutical composition, comprising a compound of formula (I) as defined in claim 15, or a deuterated or tritiated form of the compound of formula (I), or a pharmaceutically acceptable salt thereof.

24. A medicament comprising a compound of formula (I) according to claim 15, or a deuterated or tritiated form of the compound of formula (I), or a pharmaceutically acceptable salt thereof.

25. A method of treating and/or preventing in a subject in need thereof Protein tyrosine kinase mediated diseases, in particular phosphatidylinositol 3-kinase mediated diseases, more particularly of diseases mediated by the isoform a of phosphatidylinositol 3-kinase, comprising administering to the subject a compound of formula (I) according to claim 15, or a deuterated or tritiated form of the compound of formula (I), or a pharmaceutically acceptable salt thereof.

26. The method according to claim 25, wherein Protein tyrosine kinase mediated diseases, in particular phosphatidylinositol 3-kinase mediated diseases, more particularly diseases mediated by the isoform a of phosphatidylinositol 3-kinase, are selected from the group consisting of PI3K related Overgrowth Spectrum (PROS); neurofibromatosis; mitochondrial genetic diseases; keloids; hypertrophic scars, including burn scars; hyperpigmentation disorders; proliferative diseases, in particular cancers, inflammatory diseases including autoimmune disorders; and glomerulonephritis, and more particularly selected from the group consisting of keloids; hypertrophic scars, including burn scars; and hyperpigmentation disorders.

27. A compound of formula (I) according to claim 15, or a deuterated or tritiated form of the compound of formula (I), or a pharmaceutically acceptable salt thereof, for use in the treatment and/or prevention of cancer, in particular bladder cancer, breast cancer, lung cancer, such as non-small cell lung cancer and small-cell lung cancer, ovarian cancer, cervical cancer, kidney cancer, liver cancer, head and neck cancer, such as head and neck squamous cell carcinoma, sarcoma, brain cancer, such as glioma, glioblastoma, anaplastic oligodendroglioma and anaplastic astrocytoma, or hematological cancer, such as leukemia, lymphoma and myeloma.

28. A compound of formula (I) according to claim 15, or a deuterated or tritiated form of the compound of formula (I), or a pharmaceutically acceptable salt thereof, for use in the treatment and/or prevention of phosphatidylinositol 3-kinase related Overgrowth Spectrum, in particular Congenital PROS, Lipomatous, Overgrowth, Vascular Malformations, Epidermal Nevi and Spinal/Skeletal Anomalies and/or Scoliosis syndrome.
